(19) United States
(12) Patent Application Publication
(10) Pub. No.: US 2014/0107166 A1
(43) Pub. Date: Apr. 17, 2014

(54) HISTONE DEACETYLASE INHIBITORS AND METHODS OF USE THEREOF

(71) Applicants: Ralph Mazitschek, Belmont, MA (US); James E. Bradner, Cambridge, MA (US); Melissa Grachan Harman, Moreno Valley, CA (US)

(72) Inventors: Ralph Mazitschek, Belmont, MA (US); James E. Bradner, Cambridge, MA (US); Melissa Grachan Harman, Moreno Valley, CA (US)

(73) Assignee: DANA-FARBER CANCER INSTITUTE, INC., Boston, MA (US)

(21) Appl. No.: 13/985,149

(22) PCT Filed: Feb. 13, 2012
(86) PCT No.: PCT/US12/24868

§ 371 Date: Dec. 26, 2013

Related U.S. Application Data
(60) Provisional application No. 61/442,796, filed on Feb. 14, 2011.

(51) Int. Cl. C07C 259/10; A61K 31/165; C07D 213/81; C07D 333/38; C07C 69/734; C07C 259/06

(52) U.S. Cl. CPC C07C 259/10; C07C 69/734; C07C 259/06; C07D 213/81; C07D 333/38; A61K 31/165
USPC 514/354; 560/55; 514/532; 562/621; 514/575; 514/533; 562/622; 560/42; 514/538; 546/323; 549/72; 514/448; 435/375

(57) ABSTRACT

The present invention provides novel compounds for inhibiting histone deacetylases, and pharmaceutically acceptable salts and derivatives thereof. The present invention further provides methods for treating disorders regulated by histone deacetylase activity (e.g., proliferative diseases, cancer, inflammatory diseases, protozoal infections, hair loss, etc.) comprising administering a therapeutically effective amount of a compound of the invention to a subject in need thereof. The present invention also provides methods for preparing compounds of the invention.

### RELATED APPLICATIONS

This application claims the benefit of U.S. Patent Application No. 61/442,796 filed Feb. 14, 2011, the contents of which is hereby incorporated by reference in its entirety.

### STATEMENT OF RIGHTS TO INVENTIONS MADE UNDER FEDERALLY SPONSORED RESEARCH

This work was supported at least in part by the following grant: Grant No. T32CA079443. The government has certain rights in the invention.

### BACKGROUND OF THE INVENTION

Histone deacetylases have recently become the subject of considerable interest as potential targets for treatment of disease (see, for example, a discussion of the use of inhibitors of histone deacetylases for the treatment of cancer: Marks et al. Nature Reviews Cancer 2001, 7, 194; Johnstone et al. Nature Reviews Drug Discovery 2002, 1, 287; see also U.S. Pat. No. 7,250,504; U.S. Pat. No. 6,777,217; U.S. Published Application 2005/0287629; each of which is incorporated herein by reference). HDACs are zinc hydrolases that modulate gene expression through deacetylation of the N-acetyl-lysine residues of histone proteins and other transcriptional regulators (Hassig et al. Cum Opin. Chem. Biol. 1997, 1, 300-308). HDACs participate in cellular pathways that control cell shape and differentiation, and at least one HDAC inhibitor has been shown effective in treating an otherwise recalcitrant cancer (Warren et al. J. Natl. Cancer Inst. 1998, 90, 1621-1625). Eleven human HDACs, which use Zn as a cofactor, have been characterized (Taunton et al. Science 1996, 272, 408-411; Yang et al. J. Biol. Chem. 1997, 272, 28001-28007; Grozinger et al. Proc. Natl. Acad. Sci. U.S.A. 1999, 96, 4868-4873; Kao et al. Genes Dev. 2000, 14, 55-66; Hu et al. J. Biol. Chem. 2000, 275, 15254-15264; Zhou et al. Proc. Natl. Acad. Sci. U.S.A 2001, 98, 10572-10577; Venter et al. Science 2001, 291, 1304-1351). These members fall into three related classes (class I, II and IV). An additional seven HDACs have been identified which use NAD as a co-factor.

In addition, acetylation of non-histone substrates has been implicated in a variety of cellular processes, such as chromatin remodeling, cell cycle, splicing, nuclear transport, and actin nucleation (see, e.g., Choudhary, C. et al., Science (2009), 325(5942):834-840).

There remains a need for more potent and/or more specific deacetylase inhibitors (e.g., HDAC inhibitors) for treating diseases associated with aberrant deacetylase activity such as cancer, for treatment of parasitic diseases (such as malaria, sleeping sickness, Chagas disease, and the like), for treatment of inflammation, for use as mood stabilizers, for treatment of epilepsy, or for treatment of neurodegenerative diseases such as Alzheimer's disease and Huntington's disease.

### SUMMARY OF THE INVENTION

The present invention provides novel deacetylase inhibitors and methods of preparing and using these novel compounds. In certain embodiments, the deacetylase inhibitors are histone deacetylase (HDAC) inhibitors. The compounds of the invention may be useful in the treatment of proliferative diseases such as cancer, or for treatment of parasitic diseases (such as malaria, sleeping sickness, Chagas disease, and the like), for treatment of inflammation, for use as mood stabilizers, for treatment of epilepsy, or for treatment of neurodegenerative diseases such as Alzheimer's disease and Huntington's disease.

For example, in one aspect, the present invention provides novel compounds of formula (I),

CAP-L-Ar-(CR₁R₂)ₙ—Z (I)

wherein R₁ and R₂ are each independently selected from the group consisting of H, halogen, C₁-C₄alkyl, or C₁-C₄haloalkyl; Ar is a substituted or unsubstituted aryl group having from 5-14 atoms in the aryl ring(s); L is a linker; CAP is a cap; Z is a chelator capable of binding Zn²⁺; and n is 1, 2 or 3; provided that if n is 1, CAP is not carbazolyl;
or a pharmaceutically acceptable salt thereof.

In another aspect, the invention provides a compound represented by the formula:

Ar—C(O)NHOH (II)

wherein Ar is a substituted or unsubstituted aryl or heteroaryl group having from 5-14 atoms in the aryl ring(s); provided that if Ar is a phenyl group, the phenyl group is substituted; or a pharmaceutically acceptable salt thereof.

In another embodiment, the invention provides a compound represented by the formula:

CAP-L-Ar—C(O)NHOH (III)

wherein
Ar is an aryl or heteroaryl group having from 5-14 atoms in the aryl ring(s);
L is a linker;
CAP is a cap;
or a pharmaceutically acceptable salt thereof.

In certain embodiments, L is a linker having 1-6 atoms. In certain embodiments, the linker comprises an alkylidene, an ether, a thioether, an amine, an amide, an ester, a carbonate, a carbamate, or a hydrazone. In certain embodiments, the linker comprises —(CH₂)ₘ—O—, wherein m is an integer from 1 to 4.

In certain embodiments, CAP is a substituted or unsubstituted aryl group. In certain embodiments, CAP is a substituted or unsubstituted phenyl group. In certain embodiments, the phenyl group is substituted with 1-5 electron-withdrawing substituents. In certain embodiments, the 1-5 electron-withdrawing substituents are 1-5 fluorine atoms.

The invention also provides various pharmaceutically acceptable forms of the inventive compounds, for examples, stereoisomers, enantiomers, tautomers, salts, solvates, hydrates, co-crystals, and polymorphs.

In another aspect, the present invention provides pharmaceutical compositions comprising a therapeutically effective amount of a compound of formula (I), (II) or (III) and a pharmaceutically acceptable excipient. In certain embodiments, the pharmaceutical composition is useful in the treatment of a proliferative disease such as cancer.

In yet another aspect, the present invention provides methods for inhibiting deacetylase activity in a subject or a biological sample, comprising administering to said subject, or contacting said biological sample, with an amount of a compound of the invention effective to inhibit deacetylase activity in the subject or biological sample. In certain embodiments, the method is used to specifically inhibit histone deacetylase activity in the subject or biological sample. In certain embodiments, the method is used to specifically inhibit a particular histone deacetylase activity in the subject or biological sample. In certain embodiments, the compounds specifically inhibit a particular HDAC (e.g., HDAC1, HDAC2, HDAC3, HDAC4, HDAC5, HDAC6, HDAC7, HDAC8, HDAC9, HDAC10, HDAC11) or class of HDACs (e.g., Class I, II and/or IV). In certain embodiments, the inventive compound specifically inhibits HDAC6. In certain embodiments, the inventive compound specifically inhibits HDAC6 and HDAC8. In certain embodiments, the method is used to specifically inhibit tubulin deacetylase activity in a subject or a biological sample.

In certain embodiments, the present invention provides a method of treating a proliferative disease (e.g., cancer, benign neoplasm, autoimmune disease, inflammatory disease, diabetic retinopathy) comprising administering a therapeutically effective amount of a compound of formula (I), (II) or (III) to a subject with a proliferative disease. In certain embodiments, the present invention provides a method of treating cancer (e.g., such as effecting tumor cell death or inhibiting the growth of tumor cells) by administering a therapeutically effective amount of a compound of formula (I), (II) or (III) to a subject in need thereof. Exemplary cancer include, but are not limited to, breast cancer, cervical cancer, colon and rectal cancer, leukemia, lung cancer, melanoma, multiple myeloma, non-Hodgkin's lymphoma, lymphoma, ovarian cancer, pancreatic cancer, prostate cancer, skin cancer, and gastric cancer. In certain embodiments, the inventive compounds are active against leukemia cells and melanoma cells, and thus are useful for the treatment of leukemias (e.g., myeloid, lymphocytic, myelocytic and lymphoblastic leukemias) and malignant melanomas. In certain embodiments, the inventive compounds are useful in the treatment of cutaneous T-cell lymphoma (CTCL). The compounds may be administered by any method known in the art. In certain embodiments, the compounds are administered orally or parenterally (e.g., by intravenous, intraarterial, intramuscular, and/or subcutaneous injection).

In certain embodiments, the present invention provides a method of treating a parasitic disease such as malaria, sleeping sickness, Chagas disease, and the like. The method comprises administering a therapeutically effective amount of a compound of formula (I), (II) or (III) to a subject suffering from or susceptible to a parasitic disease. In certain embodiments, the present invention provides a method of treating malaria.

The inventive compounds are also useful as tools to probe biological function. The compounds may be used to probe gene expression or to elucidate biological pathways. In certain embodiments, the compounds are used as probes of signal transduction pathways.

In still another aspect, the present invention provides methods for preparing compounds of the invention and intermediates thereof.

In still another aspect, the invention provides a method of treating a subject suffering from or susceptible to a proliferative disorder or a parasitic disease, the method comprising steps of administering a therapeutically effective amount of bufexamac to the subject such that the proliferative disorder or parasitic disease is treated or prevented.

In yet another aspect, the invention provides packaged pharmaceutical comprising a therapeutically effective amount of a compound of any of Formulae (I), (II), or (III), and written instructions for administration of the compound to a subject in need thereof.

### BRIEF DESCRIPTION OF THE DRAWINGS

FIG. 1 is a scheme depicting exemplary syntheses of certain compounds of the invention.

FIG. 2 is a table showing certain compounds of the invention and their activity.

### DETAILED DESCRIPTION OF THE INVENTION

The present invention provides novel compounds of general formulae (I) and (II) and pharmaceutical compositions thereof, and methods of using the compounds.

Bufexamac is a drug approved in Europe for the topical treatment of inflammation. It is available in forms for topical administration to skin (for treatment of atopic dermatitis and other skin conditions) and for rectal administration (for treatment of hemorrhoids).

It has now been found that bufexamac is an inhibitor of HDAC. Thus, in one aspect, the present invention relates to the use of bufexamac for the inhibition of HDACs, including HDAC6 and/or HDAC8, and to derivatives and analogs of bufexamac and their use for the inhibition of HDACs, including HDAC6. Treatment of conditions related to HDAC activity is also provided.

In certain embodiments, a derivative or analog of bufexamac provides improved (more potent or selective) inhibition of at least one HDAC than does bufexamac itself. In certain embodiments, a compound according to the invention includes a “warhead” moiety capable of inhibiting an HDAC or HDACs (e.g., a zinc-binding element (ZBE), a chelator of Zn²⁺) together with a “cap” and a linker for covalently joining the warhead and the cap. Thus, in certain embodiments, the invention provides compounds generally including a metal chelating moiety connected to a cap through a linker.

In certain embodiments, the deacetylase inhibitor is a histone deacetylase (HDAC) inhibitor. In certain particular embodiments, the deacetylase inhibitor is an HDAC6 inhibitor. In certain embodiments, the deacetylase inhibitor is a tubulin deacetylase (TDAC) inhibitor. The invention also provides methods for the synthesizing and using the inventive compounds. The compounds are useful as deacetylase inhibitors (e.g. HDAC inhibitors) and are useful in the treatment of proliferative diseases, such as cancer.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic, aliphatic and heteroaliphatic, carbon and heteroatom substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example of proliferative diseases, including, but not limited to cancer. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

Certain compounds of the present invention can comprise one or more asymmetric centers, and thus can exist in various isomeric forms, e.g., stereoisomers and/or diastereomers. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds.

Where a particular enantiomer is desired, it may, in some embodiments be provided substantially free of the corresponding enantiomer, and may also be referred to as “optically enriched.” “Optically enriched,” as used herein, means that the compound is made up of a significantly greater proportion of one enantiomer. In certain embodiments the compound is made up of at least about 90% by weight of a preferred enantiomer. In other embodiments the compound is made up of at least about 95%, 96%, 97%, 98%, or 99% by weight of a desired enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et ah, Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); Wilen, Tables of Resolving Agents and Optical Resolutions p. 268 (E. L. Eliel, Ed., Univ. of Notre Dame Press, Notre Dame, Ind. 1972).

The term “acyl”, as used herein, refers to a carbonyl-containing functionality, e.g., —C(═O)R, wherein R is an aliphatic, alicyclic, heteroaliphatic, heterocyclic, aryl, heteroaryl, (aliphatic)aryl, (heteroaliphatic)aryl, heteroaliphatic(aryl) or heteroaliphatic(heteroaryl) moiety, whereby each of the aliphatic, heteroaliphatic, aryl, or heteroaryl moieties is substituted or unsubstituted, or is a substituted (e.g., hydrogen or aliphatic, heteroaliphatic, aryl, or heteroaryl moieties) oxygen or nitrogen containing functionality {e.g., forming a carboxylic acid, ester, or amide functionality).

The term “aliphatic”, as used herein, includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted, unsubstituted, branched or unbranched) having 1-6 carbon atoms.

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

The term “alicyclic”, as used herein, refers to compounds which combine the properties of aliphatic and cyclic compounds and include but are not limited to cyclic, or polycyclic aliphatic hydrocarbons and bridged cycloalkyl compounds, which are optionally substituted with one or more functional groups. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl, cyclohexyl, —CH₂-cyclohexyl, cyclohexenylethyl, cyclohexanylethyl, norborbyl moieties and the like, which again, may bear one or more substituents.

The term “alkoxy” (or “alkyloxy”), or “thioalkyl” as used herein refers to an alkyl group, as previously defined, attached to the parent molecular moiety through an oxygen atom or through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

The term “alkylamino” refers to a group having the structure —NHR′, wherein R′ is alkyl, as defined herein. The term “aminoalkyl” refers to a group having the structure NH₂R′—, wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; or —NRₓ(CO)Rₓ, wherein each occurrence of Rₓ independently includes, but is not limited to, aliphatic, alicyclic, heteroaliphatic, heterocyclic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the term “aryl”, as used herein, refers to a stable mono- or polycyclic, unsaturated moiety having preferably 3-14 carbon atoms, each of which may be substituted or unsubstituted. In certain embodiments, the term “aryl” refers to a planar ring having p-orbitals perpendicular to the plane of the ring at each ring atom and satisfying the Huckel rule where the number of pi electrons in the ring is (4n+2) wherein n is an integer. A mono- or polycyclic, unsaturated moiety that does not satisfy one or all of these criteria for aromaticity is defined herein as “non-aromatic”, and is encompassed by the term “alicyclic”.

The term “cap”, as used herein, refers to moiety capable of capping the linker group of a compound of the invention. The cap is generally a moiety having from 3-30 atoms (such as C, N, O, F and H), and can include a surface recognition domain that interacts with residues near the entrance to the HDAc active site. A cap can include an aryl or heteroaryl group, which may substituted with 1-5 electron-withdrawing substituents.

In general, the term “heteroaryl”, as used herein, refers to a stable mono- or polycyclic, unsaturated moiety having preferably 3-14 carbon atoms, each of which may be substituted or unsubstituted; and comprising at least one heteroatom selected from O, S, and N within the ring (i.e., in place of a ring carbon atom). In certain embodiments, the term “heteroaryl” refers to a planar ring comprising at least one heteroatom, having p-orbitals perpendicular to the plane of the ring at each ring atom, and satisfying the Huckel rule where the number of pi electrons in the ring is (4n+2) wherein n is an integer.

It will also be appreciated that aryl and heteroaryl moieties, as defined herein may be attached via an alkyl or heteroalkyl moiety and thus also include -(alkyl)aryl, -(heteroalkyl)aryl, -(heteroalkyl)heteroaryl, and -(heteroalkyl)heteroaryl moieties. Thus, as used herein, the phrases “aryl or heteroaryl moieties” and “aryl, heteroaryl, -(alkyl)aryl, -(heteroalkyl)aryl, -(heteroalkyl)heteroaryl, and -(heteroalkyl)heteroaryl” are interchangeable. Substituents include, but are not limited to, any of the previously mentioned substituents, i.e., the substituents recited for aliphatic moieties, or for other moieties as disclosed herein, resulting in the formation of a stable compound.

The term “aryl”, as used herein, does not differ significantly from the common meaning of the term in the art, and refers to an unsaturated cyclic moiety comprising at least one aromatic ring. In certain embodiments, “aryl” refers to a mono- or bicyclic carbocyclic ring system having one or two aromatic rings including, but not limited to, phenyl, naphthyl, tetrahydronaphthyl, indanyl, indenyl and the like.

The term “heteroaryl”, as used herein, does not differ significantly from the common meaning of the term in the art, and refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; N(Rₓ)₂; —S(O)₂Rₓ; or —NRₓ(CO)Rₓ, wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety. Additional examples of generally applicable substituents are illustrated by the specific embodiments described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of aliphatic, alicyclic, heteroaliphatic or heterocyclic moieties, may optionally be substituted with substituents including, but not limited to aliphatic; alicyclic; heteroaliphatic; heterocyclic; aromatic; hetero aromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; or —NRₓ(CO)Rₓ, wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heteroaliphatic”, as used herein, refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e.g., in place of carbon atoms. Heteroaliphatic moieties may be linear or branched, and saturated or unsaturated. In certain embodiments, heteroaliphatic moieties are substituted by independent replacement of one or more of the hydrogen atoms thereon with one or more moieties including, but not limited to aliphatic; alicyclic; heteroaliphatic; heterocyclic; aromatic; hetero aromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; or —NRₓ(CO)Rₓ, wherein each occurrence of Rx independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments described herein.

The term “heterocycloalkyl”, “heterocycle” or “heterocyclic”, as used herein, refers to compounds which combine the properties of heteroaliphatic and cyclic compounds and include, but are not limited to, saturated and unsaturated mono- or polycyclic cyclic ring systems having 5-16 atoms wherein at least one ring atom is a heteroatom selected from O, S and N (wherein the nitrogen and sulfur heteroatoms may be optionally be oxidized), wherein the ring systems are optionally substituted with one or more functional groups, as defined herein. In certain embodiments, the term “heterocycloalkyl”, “heterocycle” or “heterocyclic” refers to a non-aromatic 5-, 6- or 7-membered ring or a polycyclic group wherein at least one ring atom is a heteroatom selected from O, S, and N (wherein the nitrogen and sulfur heteroatoms may be optionally be oxidized), including, but not limited to, a bi- or tri-cyclic group, comprising fused six-membered rings having between one and three heteroatoms independently selected from oxygen, sulfur and nitrogen, wherein (i) each 5-membered ring has 0 to 2 double bonds, each 6-membered ring has 0 to 2 double bonds and each 7-membered ring has 0 to 3 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring. Representative heterocycles include, but are not limited to, heterocycles such as furanyl, thiofuranyl, pyranyl, pyrrolyl, thienyl, pyrrolidinyl, pyrazolinyl, pyrazolidinyl, imidazolinyl, imidazolidinyl, piperidinyl, piperazinyl, oxazolyl, oxazolidinyl, isooxazolyl, isoxazolidinyl, dioxazolyl, thiadiazolyl, oxadiazolyl, tetrazolyl, triazolyl, thiatriazolyl, oxatriazolyl, thiadiazolyl, oxadiazolyl, morpholinyl, thiazolyl, thiazolidinyl, isothiazolyl, isothiazolidinyl, dithiazolyl, dithiazolidinyl, tetrahydrofuryl, and benzofused derivatives thereof. In certain embodiments, a “substituted heterocycle, or heterocycloalkyl or heterocyclic” group is utilized and as used herein, refers to a heterocycle, or heterocycloalkyl or heterocyclic group, as defined above, substituted by the independent replacement of one, two or three of the hydrogen atoms thereon with, but are not limited to, aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; or —NRₓ(CO)Rₓ, wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples or generally applicable substituents are illustrated by the specific embodiments described herein.

The term “linker,” as used herein, refers to a chemical moiety utilized to covalently attach a functional group (e.g., an HDAC inhibitor functional group) to another functional group. Exemplary linkers are described herein. A linker can include, e.g., a substituted or unsubstituted, cyclic or acyclic, branched or unbranched aliphatic moiety; a substituted or unsubstituted, cyclic or acyclic, branched or unbranched heteroaliphatic moiety; a substituted or unsubstituted aryl moiety; or a substituted or unsubstituted heteroaryl moiety. It will be appreciated that other linkers that are known in the art can also be employed for the synthesis of the compounds of the invention.

It will be appreciated that any of the alicyclic or heterocyclic moieties described above and herein may comprise an aryl or heteroaryl moiety fused thereto. Additional examples of generally applicable substituents are illustrated by the specific embodiments described herein. The terms “halo” and “halogen” as used herein refer to an atom selected from fluorine, chlorine, bromine, and iodine.

The terms “halo” and “halogen” as used herein refer to an atom selected from fluorine, chlorine, bromine, and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “amino”, as used herein, refers to a primary (—NH₂), secondary (—NHRₓ), tertiary (—NRₓR_{y}), or quaternary (—N⁺RₓR_{y}R₂)amine, where Rₓ, R_{y} and R₂ are independently an aliphatic, alicyclic, heteroaliphatic, heterocyclic, aryl, or heteroaryl moiety, as defined herein. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “alkylidene”, as used herein, refers to a substituted or unsubstituted, linear or branched saturated divalent radical consisting solely of carbon and hydrogen atoms, having from one to n carbon atoms, having a free valence “-” at both ends of the radical. In certain embodiments, the alkylidene moiety has 1 to 6 carbon atoms.

The term “alkenylidene”, as used herein, refers to a substituted or unsubstituted, linear or branched unsaturated divalent radical consisting solely of carbon and hydrogen atoms, having from two to n carbon atoms (in which n is three to 20), having a free valence “-” at both ends of the radical, and wherein the unsaturation is present only as double bonds and wherein a double bond can exist between the first carbon of the chain and the rest of the molecule. In certain embodiments, the alkenylidene moiety has 2 to 6 carbon atoms.

The term “alkynylidene”, as used herein, refers to a substituted or unsubstituted, linear or branched unsaturated divalent radical consisting solely of carbon and hydrogen atoms, having from two to n carbon atoms (in which n is three to 20), having a free valence “-” at both ends of the radical, and wherein the unsaturation is present only as triple or double bonds and wherein a triple or double bond can exist between the first carbon of the chain and the rest of the molecule. In certain embodiments, the alkynylidene moiety has 2 to 6 carbon atoms.

Unless otherwise indicated, as used herein, the terms “alkyl”, “alkenyl”, “alkynyl”, “heteroalkyl”, “heteroalkenyl”, “heteroalkynyl”, “alkylidene”, alkenylidene”, -(alkyl)aryl, -(heteroalkyl)aryl, -(heteroalkyl)aryl, -(heteroalkyl)heteroaryl, and the like encompass substituted and unsubstituted, and linear and branched groups. Similarly, the terms “aliphatic”, “heteroaliphatic”, and the like encompass substituted and unsubstituted, saturated and unsaturated, and linear and branched groups. Similarly, the terms “cycloalkyl”, “heterocycle”, “heterocyclic”, and the like encompass substituted and unsubstituted, and saturated and unsaturated groups. Additionally, the terms “cycloalkenyl”, “cycloalkynyl”, “heterocycloalkenyl”, “heterocycloalkynyl”, “aromatic”, “heteroaromatic, “aryl”, “heteroaryl” and the like encompass both substituted and unsubstituted groups.

The term “electron-withdrawing group” refers to a substituent having an electronegativity greater than a methyl group. Examples of electron-withdrawing group include a halogen atom (e.g., a fluorine atom, a chlorine atom, a bromine atom, an iodine atom), a cyano group, an acyl group, an oxo group, a halogenalkyl group (e.g., a C₁-C₄haloalkyl group such as fluoromethyl, chloromethyl, bromomethyl, iodomethyl, difluoromethyl, trifluoromethyl and the like) and the like.

The phrase “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Pro-drugs of a variety of compounds, and materials and methods for derivatizing the parent compounds to create the pro-drugs, are known and may be adapted to the present invention. Pharmaceutically acceptable derivatives also include “reverse pro-drugs.” Reverse pro-drugs, rather than being activated, are inactivated upon absorption. For example, as discussed herein, many of the ester-containing compounds of the invention are biologically active but are inactivated upon exposure to certain physiological environments such as a blood, lymph, serum, extracellular fluid, etc. which contain esterase activity. The biological activity of reverse pro-drugs and pro-drugs may also be altered by appending a functionality onto the compound, which may be catalyzed by an enzyme. Also, included are oxidation and reduction reactions, including enzyme-catalyzed oxidation and reduction reactions. Certain exemplary pharmaceutical compositions and pharmaceutically acceptable derivatives will be discussed in more detail herein below.

By the term “protecting group”, has used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, as detailed herein, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM or MPM (p-methoxybenzyloxymethyl ether), to name a few), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals. In certain other exemplary embodiments, nitrogen protecting groups are utilized. These nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al, describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and quaternary ammonium salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moeity advantageously has not more than 6 carbon atoms. Examples of particular esters include formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As used herein, the term “tautomer” includes two or more interconvertable compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol; amide-to-imide; lactam-to-lactim; enamine-to-imine; and enamine-to-(a different) enamine tautomerizations.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, an isomer/enantiomer may, in some embodiments, be provided substantially free of the corresponding enantiomer, and may also be referred to as “optically enriched.” “Optically-enriched,” as used herein, means that the compound is made up of a significantly greater proportion of one enantiomer. In certain embodiments the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al, Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

“Compound”: The term “compound” or “chemical compound” as used herein can include organometallic compounds, organic compounds, metals, transitional metal complexes, and small molecules. In certain preferred embodiments, polynucleotides are excluded from the definition of compounds. In other preferred embodiments, polynucleotides and peptides are excluded from the definition of compounds. In a particularly preferred embodiment, the term compounds refers to small molecules (e.g., preferably, non-peptidic and non-oligomeric) and excludes peptides, polynucleotides, transition metal complexes, metals, and organometallic compounds.

“Small Molecule”: As used herein, the term “small molecule” refers to a non-peptidic, non-oligomeric organic compound either synthesized in the laboratory or found in nature. Small molecules, as used herein, can refer to compounds that are “natural product-like”, however, the term “small molecule” is not limited to “natural product-like” compounds. Rather, a small molecule is typically characterized in that it contains several carbon-carbon bonds, and has a molecular weight of less than 2000 g/mol, preferably less than 1500 g/mol, although this characterization is not intended to be limiting for the purposes of the present invention. Examples of “small molecules” that occur in nature include, but are not limited to, taxol, dynemicin, and rapamycin. Examples of “small molecules” that are synthesized in the laboratory include, but are not limited to, compounds described in Tan et al., (“Stereoselective Synthesis of over Two Million Compounds Having Structural Features Both Reminiscent of Natural Products and Compatible with Miniaturized Cell-Based Assays” J. Am. Chem. Soc. 120:8565, 1998; incorporated herein by reference). In certain other preferred embodiments, natural-product-like small molecules are utilized.

“Metal chelator”: As used herein, the term “metal chelator” refers to any molecule or moiety that is capable of forming a complex (i.e., “chelates”) with a metal ion. In certain exemplary embodiments, a metal chelator refers to any molecule or moiety that “binds” to a metal ion, in solution, making it unavailable for use in chemical/enzymatic reactions. In certain embodiments, the solution comprises aqueous environments under physiological conditions. Examples of metal ions include, but are not limited to, Ca²⁺, Fe³⁺, Zn²⁺, Na⁺, etc. In certain embodiments, the metal chelator binds Zn²⁺. In certain embodiments, molecules of moieties that precipitate metal ions are not considered to be metal chelators.

As used herein the term “biological sample” includes, without limitation, cell cultures or extracts thereof; biopsied material obtained from an animal (e g, mammal) or extracts thereof; and blood, saliva, urine, feces, semen, tears, or other body fluids or extracts thereof. For example, the term “biological sample” refers to any solid or fluid sample obtained from, excreted by or secreted by any living organism, including single-celled microorganisms (such as bacteria and yeasts) and multicellular organisms (such as plants and animals, for instance a vertebrate or a mammal, and in particular a healthy or apparently healthy human subject or a human subject affected by a condition or disease to be diagnosed or investigated).

The biological sample can be in any form, including a solid material such as a tissue, cells, a cell pellet, a cell extract, cell homogenates, or cell fractions; or a biopsy, or a biological fluid. The biological fluid may be obtained from any site (e.g., blood, saliva (or a mouth wash containing buccal cells), tears, plasma, serum, urine, bile, cerebrospinal fluid, amniotic fluid, peritoneal fluid, and pleural fluid, or cells therefrom, aqueous or vitreous humor, or any bodily secretion), a transudate, an exudate (e.g. fluid obtained from an abscess or any other site of infection or inflammation), or fluid obtained from a joint (e.g. a normal joint or a joint affected by disease such as rheumatoid arthritis, osteoarthritis, gout or septic arthritis).

The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates and nucleic acids generated by partial or complete fractionation of cell or tissue homogenates. Although the sample is preferably taken from a human subject, biological samples may be from any animal, plant, bacteria, virus, yeast, etc.

The term “animal”, as used herein, refers to humans as well as non-human animals, at any stage of development, including, for example, mammals, birds, reptiles, amphibians, fish, worms and single cells. Cell cultures and live tissue samples are considered to be pluralities of animals. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

### Compounds of the Invention

In one aspect, the present invention provides compounds of the formula (I):

CAP-L-Ar-(CR₁R₂)_{b}—Z (I)

wherein

R₁ and R₂ are each independently selected from the group consisting of H, halogen, C₁-C₄alkyl, or C₁-C₄haloalkyl;

Ar is a substituted or unsubstituted aryl group having from 5-14 atoms in the aryl ring(s);

L is a linker;

CAP is a cap;

Z is a chelator capable of binding Zn²⁺; and

n is 1, 2 or 3;

provided that if n is 1, CAP is not carbazolyl;

or a pharmaceutically acceptable salt thereof.

In Formula I, the moiety CAP represents the “cap”, L is the “linker” and “Ar—(CR₁R₂)ₙ—Z” is the warhead.

In certain embodiments, R₁ and R₂ are each H for each occurrence. In certain embodiments, Ar is a substituted or unsubstituted carbocyclic aryl group having from 6 to 10 atoms in the ring system. In certain embodiments, Ar is substituted or unsubstituted phenyl. In certain embodiments, Ar is phenyl substituted with L at the position on the phenyl ring para to the —(CR₁R₂)ₙ—Z moiety.

In certain embodiments, L is a substituted or unsubstituted, cyclic or acyclic, branched or unbranched aliphatic moiety. In certain embodiments, L comprises a hydrazone linker (e.g., L is —CH═N—NH—C(O)—). In certain embodiments, comprises an ether moiety. In certain embodiments, In certain embodiments, L is C₁-C₂₀ alkylidene, preferably C₁ to C₁₂ alkylidene, more preferably C₄-C₇ alkylidene. In certain embodiments, L is a substituted or unsubstituted, cyclic or acyclic, branched or unbranched heteroaliphatic moiety. In other embodiments, L comprises a substituted or unsubstituted aryl moiety. In still other embodiments, L comprises a substituted or unsubstituted heteroaryl moiety. In certain particular embodiments, L comprises a phenyl ring. In certain embodiments, L comprises multiple phenyl rings (e.g., one, two, three, or four phenyl rings. In certain embodiments, L is a linker having 1-6 atoms. In certain embodiments, the linker comprises an alkylidene, an ether, a thioether, an amine, an amide, an ester, a carbonate, a carbamate, or a hydrazone. In certain embodiments, the linker comprises —(CH₂)ₘ—O—, wherein m is an integer from 1 to 4.

In certain embodiments, CAP is a substituted or unsubstituted aryl group. In certain embodiments, CAP is a substituted or unsubstituted phenyl group. In certain embodiments, the phenyl group is substituted with 1-5 electron-withdrawing substituents. In certain embodiments, the 1-5 electron-withdrawing substituents are 1-5 fluorine atoms.

In certain embodiments, Z is —C(O)NHOH.

In formula (I), n is an integer between 1 to 3, inclusive. In certain embodiments, n is 1. In certain embodiments, n is 2. In certain embodiments, n is 3. In certain embodiments, m is 0. In certain embodiments, m is 1. In certain embodiments, m is 2. In certain embodiments, m is 3. In certain embodiments, n is 2 or 3.

In certain embodiments, L is a substituted or unsubstituted, cyclic or acyclic, branched or unbranched aliphatic moiety. In certain embodiments, L comprises a hydrazone linker. In certain embodiments, comprises an ether moiety. In certain embodiments, In certain embodiments, L is C₁-C₂₀ alkylidene, preferably C₁ to C₁₂ alkylidene, more preferably C₄-C₇ alkylidene. In certain embodiments, L is a substituted or unsubstituted, cyclic or acyclic, branched or unbranched heteroaliphatic moiety. In other embodiments, L comprises a substituted or unsubstituted aryl moiety. In still other embodiments, L comprises a substituted or unsubstituted heteroaryl moiety. In certain particular embodiments, L comprises a phenyl ring. In certain embodiments, L comprises multiple phenyl rings (e.g., one, two, three, or four phenyl rings.

In certain embodiments, the compound is represented by formula Ia:

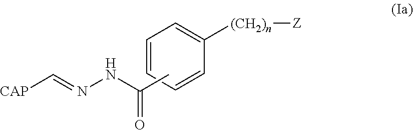

in which CAP, n and Z are as defined for Formula I. In certain embodiments, Z is —C(O)—NH(OH). In certain embodiments, CAP is the residue of an aldehyde or ketone.

In certain embodiments, the compound is represented by formula Ib:

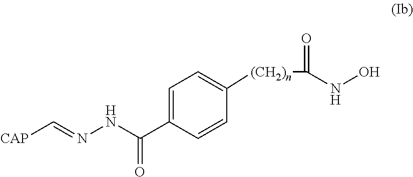

in which CAP and n are as defined for Formula I. In certain embodiments, CAP is the residue of an aldehyde or ketone.

In another aspect, the invention provides a compound represented by the formula:

Ar—C(O)NHOH (II)

wherein

Ar is a substituted or unsubstituted aryl or heteroaryl group having from 5-14 atoms in the aryl ring(s);

provided that if Ar is a phenyl group, the phenyl group is substituted;

or a pharmaceutically acceptable salt thereof.

In certain embodiments of Formula (II), Ar is a substituted phenyl group. In certain embodiments, Ar is a substituted or unsubstituted pyridyl group. In certain embodiments, Ar is a substituted or unsubstituted thiophenyl group. In certain embodiments, Ar is a phenyl, pyridyl, or thiophenyl group substituted with one to three substituents selected from the group consisting of C₁-C₆alkyl, C₂-C₆alkenyl, C₂-C₆alkynyl, C₁-C₆haloalkyl, C₁-C₆hydroxyalkyl, C₁-C₆alkoxy, C₁-C₆alkylthio, F, Cl, Br, I, —OH, —NO₂, —CN, C₁-C₆alkylsulfonyl, —C(O)C₁-C₆alkyl, —CO₂(C₁-C₆alkyl), —CON(Rₓ)₂; —OC(O)C₁-C₆alkyl; —OCO₂(C₁-C₆alkyl), —OCON(C₁-C₆alkyl)₂; —N(C₁-C₆alkyl)₂, and —NC₁-C₆alkyl(CO)C₁-C₆alkyl. In certain embodiments, Ar is substituted with one substituent. In certain embodiments, Ar is substituted with one substituent at a position ortho or para to the hydroxamate group. In certain embodiments, at least one substituent is an electron-withdrawing group. In certain embodiments, at least one substituent is selected from the group consisting of —NO₂, —CN, or F.

In another aspect, the invention provides a compound represented by the formula:

CAP-L-Ar—C(O)NHOH (III)

wherein
Ar is a substituted or unsubstituted aryl or heteroaryl group having from 5-14 atoms in the aryl ring(s);
L is a linker; and
CAP is a cap;
   or a pharmaceutically acceptable salt thereof.

In certain embodiments of Formula (III), Ar is a substituted phenyl group. In certain embodiments, Ar is a substituted or unsubstituted pyridyl group. In certain embodiments, Ar is a substituted or unsubstituted thiophenyl group. In certain embodiments, Ar is a phenyl, pyridyl, or thiophenyl group substituted with one to three substituents selected from the group consisting of C₁-C₆alkyl, C₂-C₆alkenyl, C₂-C₆alkynyl, C₁-C₆haloalkyl, C₁-C₆hydroxyalkyl, C₁-C₆alkoxy, C₁-C₆alkylthio, F, Cl, Br, I, —OH, —NO₂, —CN, C₁-C₆alkylsulfonyl, —C(O)C₁-C₆alkyl, —CO₂(C1-C₆alkyl), —CON(Rₓ)₂; —OC(O)C₁-C₆alkyl; —CO₂(C₁-C₆alkyl), —OCON(C₁-C₆alkyl)₂; —N(C₁-C₆alkyl)₂, and —NC₁-C₆alkyl(CO)C₁-C₆alkyl. In certain embodiments, Ar is substituted with one substituent. In certain embodiments, Ar is substituted with one substituent at a position ortho or para to the hydroxamate group. In certain embodiments, at least one substituent is an electron-withdrawing group. In certain embodiments, at least one substituent is selected from the group consisting of —NO₂, —CN, or F.

In certain embodiments, L is a substituted or unsubstituted, cyclic or acyclic, branched or unbranched aliphatic moiety. In certain embodiments, L comprises a hydrazone linker (e.g., L is —CH═N—NH—C(O)—). In certain embodiments, L comprises an ether moiety. In certain embodiments, L is C₁-C₂₀ alkylidene, preferably C₁ to C₁₂ alkylidene, more preferably C₄-C₇ alkylidene. In certain embodiments, L is a substituted or unsubstituted, cyclic or acyclic, branched or unbranched heteroaliphatic moiety. In other embodiments, L comprises a substituted or unsubstituted aryl moiety. In still other embodiments, L comprises a substituted or unsubstituted heteroaryl moiety. In certain particular embodiments, L comprises a phenyl ring. In certain embodiments, L comprises multiple phenyl rings (e.g., one, two, three, or four phenyl rings. In certain embodiments, L is a linker having 1-6 atoms. In certain embodiments, the linker comprises an alkylidene, an ether, a thioether, an amine, an amide, an ester, a carbonate, a carbamate, or a hydrazone. In certain embodiments, the linker comprises —(CH₂)ₘ—O—, wherein m is an integer from 1 to 4.

In certain embodiments, CAP is a substituted or unsubstituted aryl group. In certain embodiments, CAP is a substituted or unsubstituted phenyl group. In certain embodiments, the phenyl group is substituted with 1-5 electron-withdrawing substituents. In certain embodiments, the 1-5 electron-withdrawing substituents are 1-5 fluorine atoms.

In certain embodiments, L is a substituted or unsubstituted, cyclic or acyclic, branched or unbranched aliphatic moiety. In certain embodiments, L comprises a hydrazone linker. In certain embodiments, comprises an ether moiety. In certain embodiments, In certain embodiments, L is C₁-C₂₀ alkylidene, preferably C₁ to C₁₂ alkylidene, more preferably C₄-C₇ alkylidene. In certain embodiments, L is a substituted or unsubstituted, cyclic or acyclic, branched or unbranched heteroaliphatic moiety. In other embodiments, L comprises a substituted or unsubstituted aryl moiety. In still other embodiments, L comprises a substituted or unsubstituted heteroaryl moiety. In certain particular embodiments, L comprises a phenyl ring. In certain embodiments, L comprises multiple phenyl rings (e.g., one, two, three, or four phenyl rings.

In certain embodiments, the compound is represented by formula Ma:

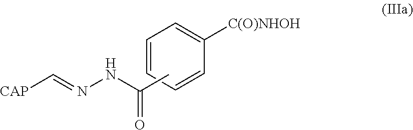

in which CAP, n and Z are as defined for Formula I. In certain embodiments, Z is —C(O)—NH(OH). In certain embodiments, CAP is the residue of an aldehyde or ketone.

In certain embodiments, the compound is represented by formula IIIb:

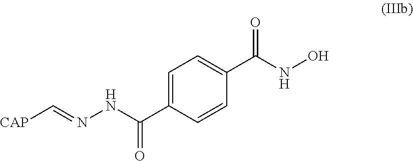

Some of the foregoing compounds can comprise one or more asymmetric centers, and thus can exist in various isomeric forms, e.g., stereoisomers and/or diastereomers. Thus, inventive compounds and pharmaceutical compositions thereof may be in the form of an individual enantiomer, diastereomer, or geometric isomer, or may be in the form of a mixture of stereoisomers. In certain embodiments, the compounds of the invention are enantiopure compounds. In certain other embodiments, mixtures of stereoisomers or diastereomers are provided.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds of the invention may be prepared by crystallization of the compound under different conditions and may exist as one or a combination of polymorphs of the compound forming part of this invention. For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other techniques. Thus, the present invention encompasses inventive compounds, their derivatives, their tautomeric forms, their stereoisomers, their polymorphs, their pharmaceutically acceptable salts their pharmaceutically acceptable solvates and pharmaceutically acceptable compositions containing them.

### Synthesis of Compounds

As would be appreciated by one of skill in the art of organic chemistry, the compounds of the invention may be prepared by any number of synthetic routes. In certain embodiments, a compound of formula (I) is prepared via condensation of a phenol-substituted aliphatic ester and a benzylic compound such as benzyl bromide, or by use of a Mitsonobu reaction of a benzylic alcohol.

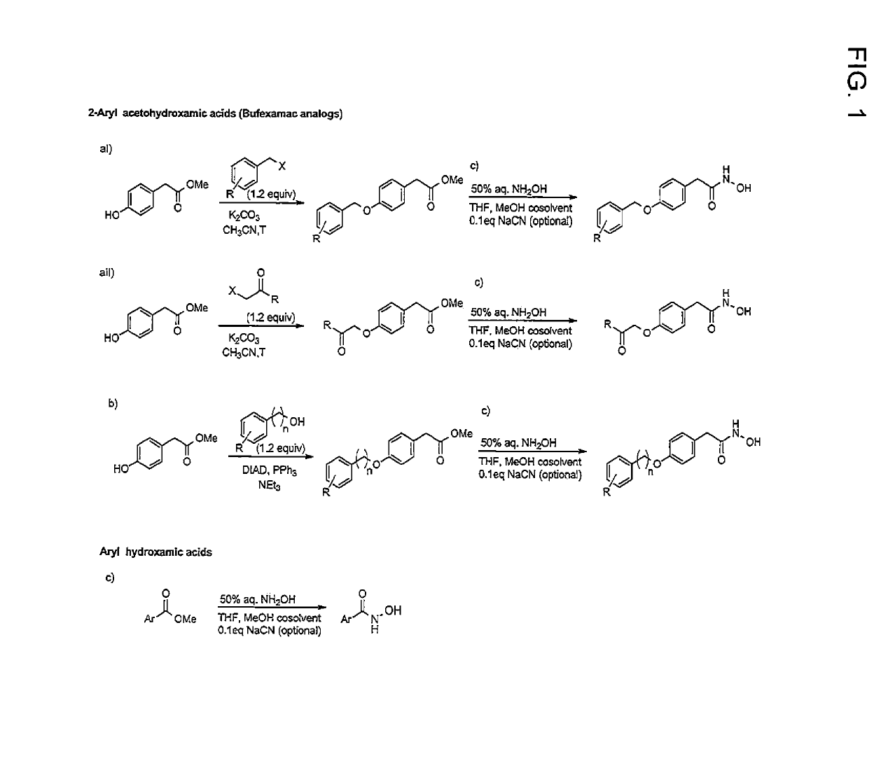

As also shown in FIG. 1, an aryl hydroxamate of the invention (e.g., of Formula (II)) can be prepared by reaction of an appropriate aryl ester with hydroxylamine.

One of ordinary skill in the art will appreciate that a wide variety of reaction conditions may be employed to promote each of the synthetic transformations as depicted in FIG. 1; therefore, a wide variety of reaction conditions are envisioned (see generally, March's Advanced Organic Chemistry: Reactions, Mechanisms, and Structure, M. B. Smith and J. March, 5th Edition, John Wiley & Sons, 2001; and Comprehensive Organic Transformations, R. C. Larock, 2nd Edition, John Wiley & Sons, 1999). In certain embodiments, the reaction is performed in an organic solvent. In certain embodiments, the organic solvent is a polar aprotic solvent. In certain embodiments, the reaction is performed in tetrahydrofuran (THF), acetonitrile, or a mixture of THF or acetonitrile and another polar aprotic solvent, such as dimethylsulfoxide or dimethylformamide.

In certain embodiments, the reaction is performed at a temperature between about 40° C. to about 80° C.

In certain embodiments, the synthetic method is amenable to high-throughput techniques or to techniques commonly used in combinatorial chemistry. Therefore, in certain embodiments, a library of compounds of Formula (I), (II), or (III) is prepared by using a variety of phenols and/or benzylic compounds in multiple reaction vessels.

### Methods of Treatment

In general, the compounds of the invention are inhibitors of deacetylase activity.

The compounds may inhibit histone deacetylase, tubulin deacetylase, or other deacetylase activity. In certain embodiments, the compounds of the invention are inhibitors of histone deacetylases (HDAC) and, as such, are useful in the treatment of disorders modulated by histone deacetylases. For example, in certain embodiments, the present invention provides a method for inhibiting deacetylase activity in a biological sample or a subject, which method comprises administering to the subject, or contacting the biological sample, an effective amount of an inventive compound or a composition thereof.

In certain embodiments, the deacetylase activity is histone deacetylase activity. In certain embodiments, the compounds specifically inhibit a particular histone deacetylase (HDAC) (e.g., HDAC1, HDAC2, HDAC3, HDAC4, HDAC4, HDAC5, HDAC6, HDAC7, HDAC8, HDAC9, HDAC10, HDAC11) or a class of HDACs (e.g., Class I, II and/or IV). In certain embodiments, the compound specifically inhibits HDAC1. In certain embodiments, the compound specifically inhibits HDAC2. In certain embodiments, the compound specifically inhibits HDAC6. In certain embodiments, the compound specifically inhibits HDAC6 and HDAC8.

In certain embodiments, the deacetylase activity is tubulin deacetylase activity. In certain embodiments, the compound of the invention is an inhibitor of a tubulin deacetylase (TDAC) and, as such, are useful in the treatment of a disorder or disease modulated by a tubulin deacetylase.

In another aspect, the present invention provides a method of treating a proliferative disease comprising administering a therapeutically effective amount of a compound of Formula (I), (II) or (III) to a subject in need thereof. In certain embodiments, the proliferative disease is associated with aberrant histone deacetylase activity and/or is controlled by modulating histone deacetylase activity. In certain embodiments, the proliferative disease is associated with aberrant tubulin deacetylase activity and/or is controlled by modulating tubulin deacetylase activity.

A subject may be any animal. In certain embodiments, the subject is any mammal (e.g., humans, domestic/veterinary/farm animals such as dogs, cats, cows, sheep, etc.). In certain embodiments, the subject is a human (e.g., child, juvenile, adult, male, female). In certain embodiments, the subject is an experimental animal such as a mouse, rat, dog, or non-human primate.

Exemplary proliferative diseases include, but are not limited to, cancer (e.g., glioblastoma, retinoblastoma, breast cancer, cervical cancer, colon and rectal cancer, leukemias, lymphoma, lung cancer (including, but not limited to small cell lung cancer and non-small cell lung cancer), melanoma and/or other skin cancers, multiple myeloma, non-Hodgkin's lymphoma, cutaneous T-cell lymphoma, and other lymphomas, ovarian cancer, pancreatic cancer, prostate cancer, gastric cancer, bladder cancer, uterine cancer, kidney cancer, testicular cancer, stomach cancer, brain cancer, liver cancer, and esophageal cancer), benign neoplasms, inflammatory disease, and autoimmune diseases.

In certain embodiments, the inventive compounds are also active against leukemia cells (e.g., leukemia cells and melanoma cells) and thus are useful for the treatment of leukemias (e.g., myeloid, lymphocytic, myelocytic and lymphoblastic leukemias). In certain embodiments, the inventive compounds are useful in the treatment of cutaneous T-cell lymphoma (CTCL) and skin cancers (e.g., squamous cell carcinoma, basal cell carcinoma, malignant melanoma, etc.). In certain embodiments, the inventive compounds are useful in the treatment of multiple myeloma. In certain embodiments, the inventive compounds are useful in the treatment of malignant melanoma.

For example, in certain embodiments, the present invention provides a method of treating cancer comprising administering a therapeutically effective amount of a compound of formula (I), (II) or (III) to a subject in need thereof. In certain embodiments, the cancer is glioblastoma, retinoblastoma, breast cancer, cervical cancer, colon and rectal cancer, leukemia, lymphoma, lung cancer (including, but not limited to small cell lung cancer and non-small cell lung cancer), melanoma and/or other skin cancers, multiple myeloma, non-Hodgkin's lymphoma, ovarian cancer, pancreatic cancer, prostate cancer and gastric cancer, bladder cancer, uterine cancer, kidney cancer, testicular cancer, stomach cancer, brain cancer, liver cancer, or esophageal cancer.

The inventive compounds are also useful in the treatment of other diseases or condition that benefit from inhibition of deacetylation activity (e.g., HDAC or TDAC inhibition), such as certain skin and/or hair conditions (e.g., psoriasis, dermatitis, hair loss, neurofibromatosis, diseases associated with skin pigmentation).

In certain embodiments, the compounds are useful in treating inflammatory diseases of the skin such as psoriasis or dermatitis.

In certain embodiments, the compounds are useful in the treatment of neurofibromatosis.

A therapeutically effective amount of an inventive compound comprises administering an amounts and for such time as is necessary to achieve a desired result. The exact amount required will vary from subject to subject, depending on the species, age, and general condition of the subject, the severity of the disease, the particular anticancer agent, its mode of administration, the desired outcome, and the like.

In certain embodiments of the present invention a “therapeutically effective amount” of the inventive compound or pharmaceutical composition is that amount effective for inhibiting deacetylase activity (e.g., HDAC and/or TDAC activity) in a subject or a biological sample (e.g., in cells). In certain embodiments, a particular deacetylase activity (e.g., HDAC1, HDAC2, HDAC3, HDAC4, HDAC4, HDAC5, HDAC6, HDAC7, HDAC8, HDAC9, HDAC10, HDAC11) is inhibited by about 10%, about 20%, about 30%, about 40%, about 50%, about 60%, about 70%, about 80%, about 90%, about 95%, or about 99%. In certain embodiments, the inventive compound inhibits HDAC6, and a therapeutically effective amount of the compound inhibits HDAC6 by at least about 25%, at least about 50%, at least about 75%, or at least about 90%.

In certain embodiments of the present invention, a “therapeutically effective amount” refers to an amount of a compound or composition sufficient to inhibit cell proliferation, or refers to a sufficient amount to reduce the effects of the proliferative disease.

In certain embodiments of the present invention a “therapeutically effective amount” of the inventive compound or pharmaceutical composition is that amount effective for reducing or inhibiting the growth of tumor cells and/or killing tumor cells.

For example, in certain embodiments, the compounds are useful in the treatment of baldness based on the discovery that HDAC inhibition (particularly, HDAC6 inhibition) blocks androgen signaling via hsp90. HDAC inhibition has also been shown to inhibit estrogen signaling.

Additionally, in certain embodiments, the present invention provides methods of treating protozoal infections comprising administering a therapeutically effective amount of a compound of formula (I), (II) or (III) to a subject in need thereof.

In certain embodiments, the inventive compounds also find use in the prevention of restenosis of blood vessels subject to traumas such as angioplasty and stenting. For example, it is contemplated that the compounds of the invention will be useful as a coating for implanted medical devices, such as tubings, shunts, catheters, artificial implants, pins, electrical implants such as pacemakers, and especially for arterial or venous stents, including balloon-expandable stents. In certain embodiments inventive compounds may be bound to an implantable medical device, or alternatively, may be passively adsorbed to the surface of the implantable device. In certain other embodiments, the inventive compounds may be formulated to be contained within, or, adapted to release by a surgical or medical device or implant, such as, for example, stents, sutures, indwelling catheters, prosthesis, and the like. For example, drugs having antiproliferative and anti-inflammatory activities have been evaluated as stent coatings, and have shown promise in preventing restenosis (See, for example, Presbitero P. et al., “Drug eluting stents do they make the difference?”, Minerva Cardioangiol, 2002, 50(5):431-442; Ruygrok P. N. et al, “Rapamycin in cardiovascular medicine”, Intern. Med. J, 2003, 33(3):103-109; and Marx S. O. et al, “Bench to bedside: the development of rapamycin and its application to stent restenosis”, Circulation, 2001, 104(8):852-855, each of these references is incorporated herein by reference in its entirety).

Accordingly, without wishing to be bound to any particular theory, Applicant proposes that inventive compounds having antiproliferative effects can be used as stent coatings and/or in stent drug delivery devices, inter alia for the prevention of restenosis or reduction of restenosis rate. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, poly(lactic-co-glycolic acid), polyglycolic acid, ethylene vinyl acetate, and mixtures thereof. The coatings may optionally be further covered by a suitable topcoat of fluorosilicone, polysaccarides, polyethylene glycol, phospholipids, or combinations thereof to impart controlled release characteristics in the composition. A variety of compositions and methods related to stent coating and/or local stent drug delivery for preventing restenosis are known in the art (see, for example, U.S. Pat. Nos. 6,517,889; 6,273,913; 6,258,121; 6,251,136; 6,248,127; 6,231,600; 6,203,551; 6,153,252; 6,071,305; 5,891,507; 5,837,313; and published U.S. patent application US2001/0027340, each of which is incorporated herein by reference in its entirety). For example, stents may be coated with polymer-drug conjugates by dipping the stent in polymer-drug solution or spraying the stent with such a solution. In certain embodiment, suitable materials for the implantable device include biocompatible and nontoxic materials, and may be chosen from the metals such as nickel-titanium alloys, steel, or biocompatible polymers, hydrogels, polyurethanes, polyethylenes, ethylenevinyl acetate copolymers, etc. In certain embodiments, the inventive compound is coated onto a stent for insertion into an artery or vein following balloon angioplasty.

The compounds of this invention or pharmaceutically acceptable compositions thereof may also be incorporated into compositions for coating implantable medical devices, such as prostheses, artificial valves, vascular grafts, stents and catheters. Accordingly, the present invention, in another aspect, includes a composition for coating an implantable device comprising a compound of the present invention as described generally above, and in classes and subclasses herein, and a carrier suitable for coating said implantable device. In still another aspect, the present invention includes an implantable device coated with a composition comprising a compound of the present invention as described generally above, and in classes and subclasses herein, and a carrier suitable for coating said implantable device.

Within other aspects of the present invention, methods are provided for expanding the lumen of a body passageway, comprising inserting a stent into the passageway, the stent having a generally tubular structure, the surface of the structure being coated with (or otherwise adapted to release) an inventive compound or composition, such that the passageway is expanded. In certain embodiments, the lumen of a body passageway is expanded in order to eliminate a biliary, gastrointestinal, esophageal, tracheal/bronchial, urethral, and/or vascular obstruction.

Methods for eliminating biliary, gastrointestinal, esophageal, tracheal/bronchial, urethral and/or vascular obstructions using stents are known in the art. The skilled practitioner will know how to adapt these methods in practicing the present invention. For example, guidance can be found in U.S. Patent Application Publication 2003/0004209 in paragraphs [0146]-[0155], which paragraphs are hereby incorporated herein by reference.

### Pharmaceutical Compositions

In another aspect, the present invention provides pharmaceutical compositions comprising a compound of formula (I), (II), or (III), or a pharmaceutically acceptable form thereof, and a pharmaceutically acceptable excipient. In certain embodiments, a therapeutically effective amount of the inventive compound is included in the pharmaceutical composition.

It will also be appreciated that certain of the compounds of present invention can exist in free form for treatment, or where appropriate, as a pharmaceutically acceptable form thereof. According to the present invention, a pharmaceutically acceptable form includes, but is not limited to, pharmaceutically acceptable salts, esters, salts of such esters, or a prodrug or other adduct or derivative of a compound of this invention which upon administration to a subject in need is capable of providing, directly or indirectly, a compound as otherwise described herein, or a metabolite or residue thereof.

As described above, the pharmaceutical compositions of the present invention comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants, and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose, and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S. P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The present invention encompasses pharmaceutically acceptable topical formulations of inventive compounds. The term “pharmaceutically acceptable topical formulation”, as used herein, means any formulation which is pharmaceutically acceptable for intradermal administration of a compound of the invention by application of the formulation to the epidermis. In certain embodiments of the invention, the topical formulation comprises a carrier system. Pharmaceutically effective carriers include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerine, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in the formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al, Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, 111. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

In certain embodiments, the compositions may be in the form of ointments, pastes, creams, lotions, gels, powders, solutions, sprays, inhalants or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

The compounds of the invention are preferably formulated in dosage unit form for ease of administration and uniformity of dosage. The expression “dosage unit form” as used herein refers to a physically discrete unit of therapeutic agent appropriate for the subject to be treated. It will be understood, however, that the total daily usage of the compounds and compositions of the present invention will be decided by the attending physician within the scope of sound medical judgment. The specific therapeutically effective dose level for any particular subject or organism will depend upon a variety of factors including the disorder being treated and the severity of the disorder; the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts (see, for example, Goodman and Gilman's The Pharmacological Basis of Therapeutics, Tenth Edition, A. Gilman, J. Hardman and L. Limbird, eds., McGraw-Hill Press, 155-173, 2001, which is incorporated herein by reference in its entirety).

Furthermore, after formulation with an appropriate pharmaceutically acceptable excipient in a desired dosage, the pharmaceutical compositions of this invention can be administered to humans and other animals orally, rectally, parenterally, intracisternally, intravaginally, intraperitoneally, intravenously, intraarterially, intramuscularly, subcutaneously, topically (as by powders, ointments, creams or drops), bucally, as an oral or nasal spray, or the like, depending on the severity of the infection being treated. In certain embodiments, the inventive compounds is administered orally or intravenously.

In certain embodiments, the compounds of the invention may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another immunomodulatory agent, anticancer agent or agent useful for the treatment of psoriasis), or they may achieve different effects {e.g., control of any adverse effects). For example, other therapies or anticancer agents that may be used in combination with the inventive compounds of the present invention include surgery, radiotherapy (in but a few examples, γ-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, The Merck Manual, Seventeenth Ed. 1999, the entire contents of which are hereby incorporated by reference. See also the National Cancer Institute (CNI) website (www.nci.nih.gov) and the Food and Drug Administration (FDA) website for a list of the FDA approved oncology drugs (www. fda.gov/cder/cancer/druglistframe).

In certain embodiments, the pharmaceutical compositions of the present invention further comprise one or more additional therapeutic agents {e.g., chemotherapeutic and/or palliative). For example, additional therapeutic agents for conjoint administration or inclusion in a pharmaceutical composition with a compound of this invention may be an approved chemotherapeutic agent and/or palliative agent, or it may be any one of a number of agents undergoing approval in the Food and Drug Administration. For purposes of the invention, the term “palliative” refers to treatment that is focused on the relief of symptoms of a disease and/or side effects of a therapeutic regimen, but is not curative. For example, palliative treatment encompasses painkillers, antinausea medications and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

### Kits

In certain embodiments, the present invention provides kits for conveniently and effectively carrying out the methods in accordance with the present invention. In general, the pack or kit comprises one or more containers filled with one or more of the ingredients of the pharmaceutical compositions of the invention. Optionally associated with such container(s) can be instructions for administration, including dosage recommendations, and/or a notice in the form prescribed by a governmental agency regulating the manufacture, use or sale of pharmaceutical products, wherein the notice reflects approval by the agency of manufacture, use or sale for human administration. A kit may include multiple dosage units. For example, a kit may include dosage units for multiple days, weeks, or months. In certain embodiments, the kit include a week-supply of the inventive compound or composition thereof. In certain embodiments, the kit include a month-supply of the inventive compound or composition thereof.

### Research Uses and Assays

The inventive compounds are also useful as tools to probe biological function. For example, the inventive compounds may be used to probe biological pathways that depend on deacetylase (e.g., HDAC) activity. In certain embodiments, the inventive compounds may be used to probe gene expression. In certain embodiments, the inventive compounds may be used to probe autophagy.

For example, according to the present invention, the inventive compounds may be assayed in any of the available assays known in the art for identifying compounds having antiprotozoal, HDAC inhibitory, hair growth, androgen signalling inhibitory, estrogen signaling inhibitory, autophagy inhibitory, and/or antiproliferative activity. For example, the assay may be cellular or non-cellular, in vivo or in vitro, high- or low-throughput format, etc.

Thus, in one aspect, compounds of this invention which are of particular interest include those which: exhibit HDAC-inhibitory activity; exhibit HDAC Class I inhibitory activity (e.g., HDAC1, HDAC2, HDAC3, HDAC8); exhibit HDAC Class II inhibitory activity (e.g., HDAC4, HDAC5, HDAC6, HDAC7, HDAC9a, HDAC9b, HDRP/HDAC9c, HDAC10); exhibit HDAC Class IV inhibitory activity; exhibit the ability to inhibit HDAC1 (Genbank Accession No. NP 004955, incorporated herein by reference); exhibit the ability to inhibit HDAC2 (Genbank Accession No. NP OO1 518, incorporated herein by reference); exhibit the ability to inhibit HDAC3 (Genbank Accession No. 015739, incorporated herein by reference); exhibit the ability to inhibit HDAC4 (Genbank Accession No. AAD29046, incorporated herein by reference); exhibit the ability to inhibit HDAC5 (Genbank Accession No. NP_{—}005465, incorporated herein by reference); exhibit the ability to inhibit HDAC6 (Genbank Accession No. NP_{—}006035, incorporated herein by reference); exhibit the ability to inhibit HDAC7 (Genbank Accession No. AAP63491, incorporated herein by reference); exhibit the ability to inhibit HDAC8 (Genbank Accession No. AAF73428, NM O1 8486, AF245664, AF230097, each of which is incorporated herein by reference); exhibit the ability to inhibit HDAC9 (Genbank Accession No. NM_{—}178425, NM_{—}178423, NM_{—}058176, NM014707, BC1_{—}11735, NM_{—}058177, each of which is incorporated herein by reference); exhibit the ability to inhibit HDAC10 (Genbank Accession No. NM032019, incorporated herein by reference); exhibit the ability to inhibit HDAC11 (Genbank Accession No. BC009676, incorporated herein by reference); exhibit the ability to inhibit tubulin deacetylation (TDAC); exhibit the ability to modulate the glucose-sensitive subset of genes downstream of Ure2p; exhibit cytotoxic or growth inhibitory effect on cancer cell lines maintained in vitro or in animal studies using a scientifically acceptable cancer cell xenograft model; and/or exhibit a therapeutic profile (e.g., optimum safety and curative effect) that is superior to existing chemotherapeutic agents.

As detailed in the exemplification herein, in assays to determine the ability of compounds to inhibit cancer cell growth certain inventive compounds may exhibit IC₅₀ values <100 μM. In certain other embodiments, inventive compounds exhibit IC₅₀ values <50 μM. In certain other embodiments, inventive compounds exhibit IC₅₀ values <40 μM. In certain other embodiments, inventive compounds exhibit IC₅₀ values <30 μM. In certain other embodiments, inventive compounds exhibit IC₅₀ values <20 μM. In certain other embodiments, inventive compounds exhibit IC₅₀ values <10 μM. In certain other embodiments, inventive compounds exhibit IC₅₀ values <7.5 μM. In certain embodiments, inventive compounds exhibit IC₅₀ values <5 μM. In certain other embodiments, inventive compounds exhibit IC₅₀ values <2.5 μM. In certain embodiments, inventive compounds exhibit IC₅₀ values <1 μM. In certain embodiments, inventive compounds exhibit IC₅₀ values <0.75 μM. In certain embodiments, inventive compounds exhibit IC₅₀ values <0.5 μM. In certain embodiments, inventive compounds exhibit IC₅₀ values <0.25 μM. In certain embodiments, inventive compounds exhibit IC₅₀ values <0.1 μM. In certain other embodiments, inventive compounds exhibit IC₅₀ values <75 nM. In certain other embodiments, inventive compounds exhibit IC₅₀ values <50 nM. In certain other embodiments, inventive compounds exhibit IC₅₀ values <25 nM. In certain other embodiments, inventive compounds exhibit IC₅₀ values <10 nM. In other embodiments, exemplary compounds exhibited IC₅₀ values <7.5 nM. In other embodiments, exemplary compounds exhibited IC₅₀ values <5 nM.

### Kits or Pharmaceutical Systems

The present compositions may be assembled into kits or pharmaceutical systems for use in ameliorating a proliferative or inflammatory disease. Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the agents of the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Synthesis of Exemplary Compounds for Use as HDAC Inhibitors

### General Description of Synthetic Methods

Certain compounds of the invention of Formula (I) containing an ether linker between the cap (CAP) and the aryl group Ar can be prepared from the starting phenol as follows:

a) 1.2 equivalent of potassium carbonate were added to a solution of 1 equivalent of the phenol in acetonitrile followed by the addition of 1.2 equivalent of the benzyl halide. The reaction mixture was stirred at 70 C. Reaction progress was monitored by LC/MS. Upon reaction of all phenol starting material (usually over night) brine was added and the aqueous solution was extracted three times with ethyl acetate. The combined organic layers were dried over sodium sulfate and concentrated under reduced pressure. The crude product, if require, was further purified on silica.
b) 1 equivalent of DIAD was added at 0° C. to a solution of 1 equivalent of the phenol, 1.2 equivalent of the alcohol, 1 equivalent triphenylphosphine and 1 equivalent triethylamine in THF. The reaction mixture was then warmed to room temperature and allowed to stir over night. Reaction progress was monitored by LC/MS. Upon reaction of all phenol starting material 1N HCl was added and the aqueous solution was extracted three times with diethyl ether. The combined organic layers were dried over sodium sulfate and concentrated under reduced pressure. The crude product, if require, was further purified on silica.
c) 1 solvent volume equivalent (less if solubility limit of starting material is reached) of 50% aqueous hydroxylamine solution was added to a solution of the alkyl ester in methanol or methanol/THF 1:1 followed by a catalytic amount (generally 10 mol %) of sodium cyanide. The reaction mixture was stirred at room temperature all starting material had been consumed as determined by LC/MS (usually 3h-24h).
Workup A: If the desired product precipitated out, the product was filtered of and washed with water. Precipitating product was generally of very high purity and did not require additional purification.
Workup B: If the desired product did not crash out, brine was added and the aqueous solution was extracted multiple times with 20% isopropanol in dichloromethane. The combined organic layers were dried over sodium sulfate and concentrated under reduced pressure. The crude product was further purified on silica or if needed by reversed phase HPLC.

The following compounds were prepared according to the general methods described above, or by other methods readily available to a person of ordinary skill in the art:

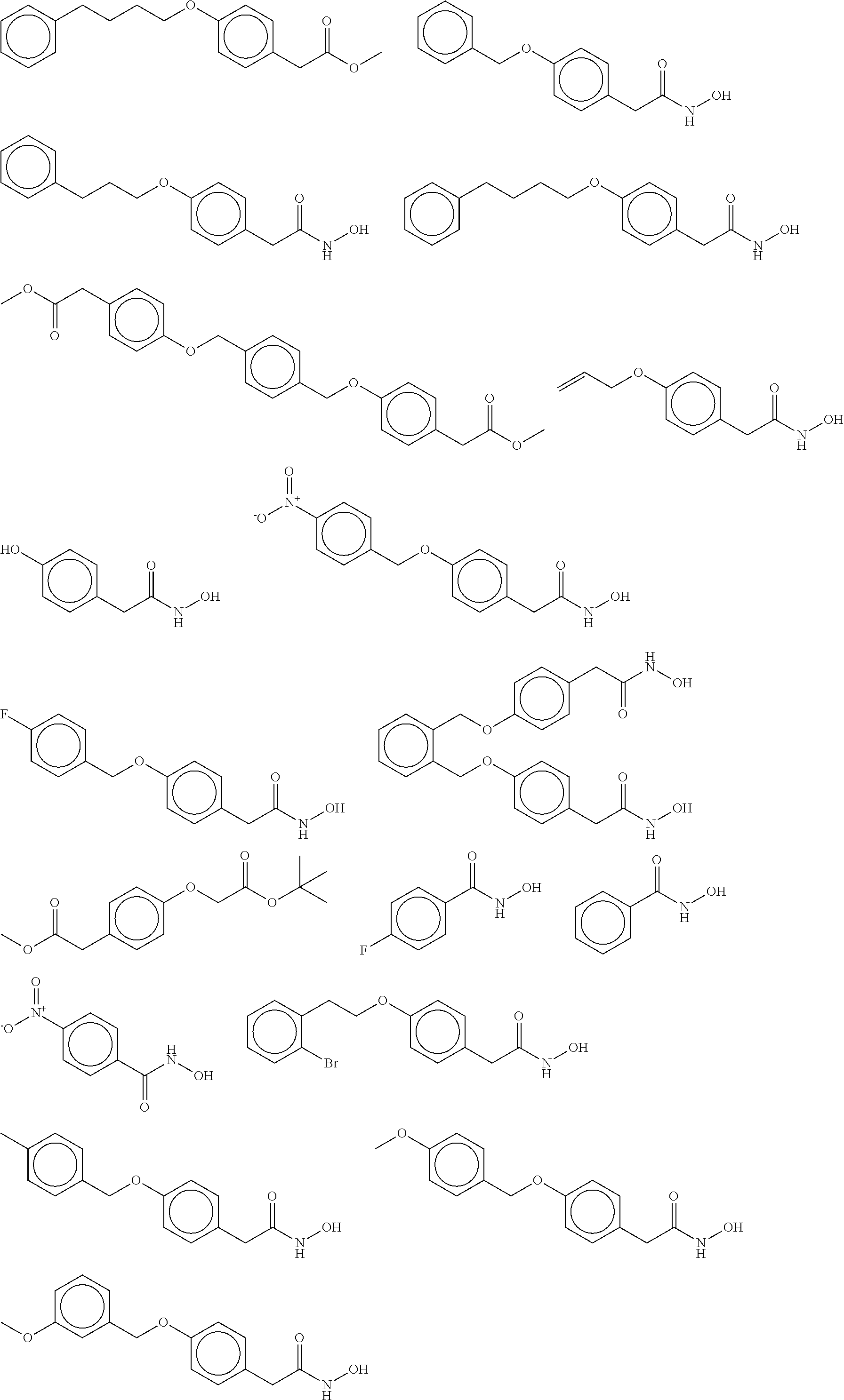

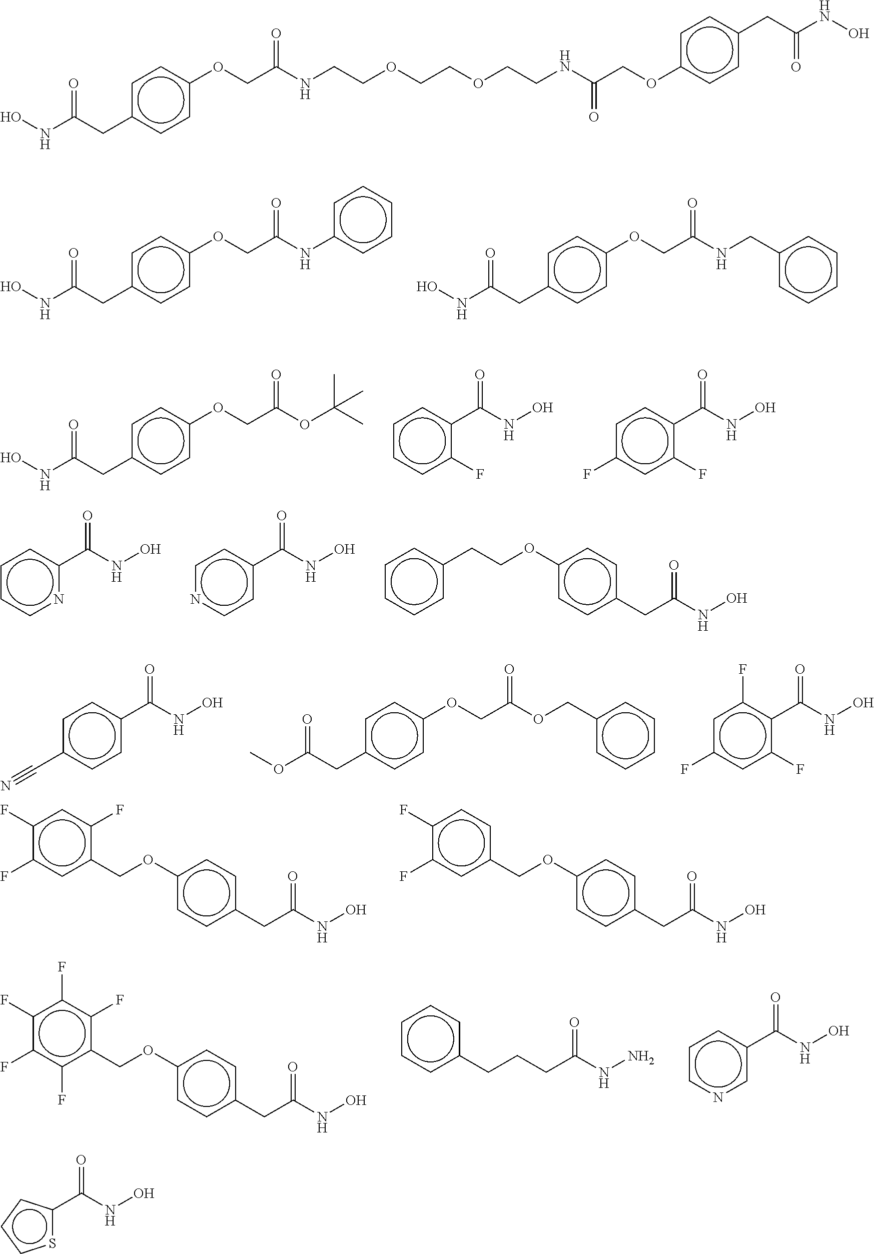

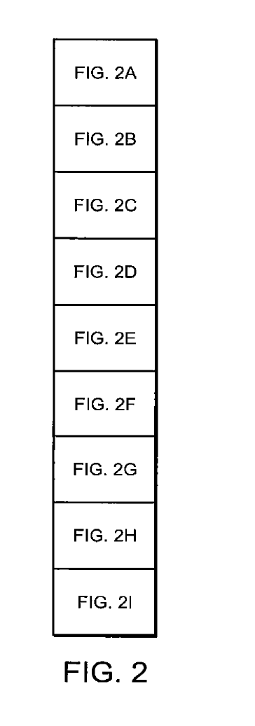

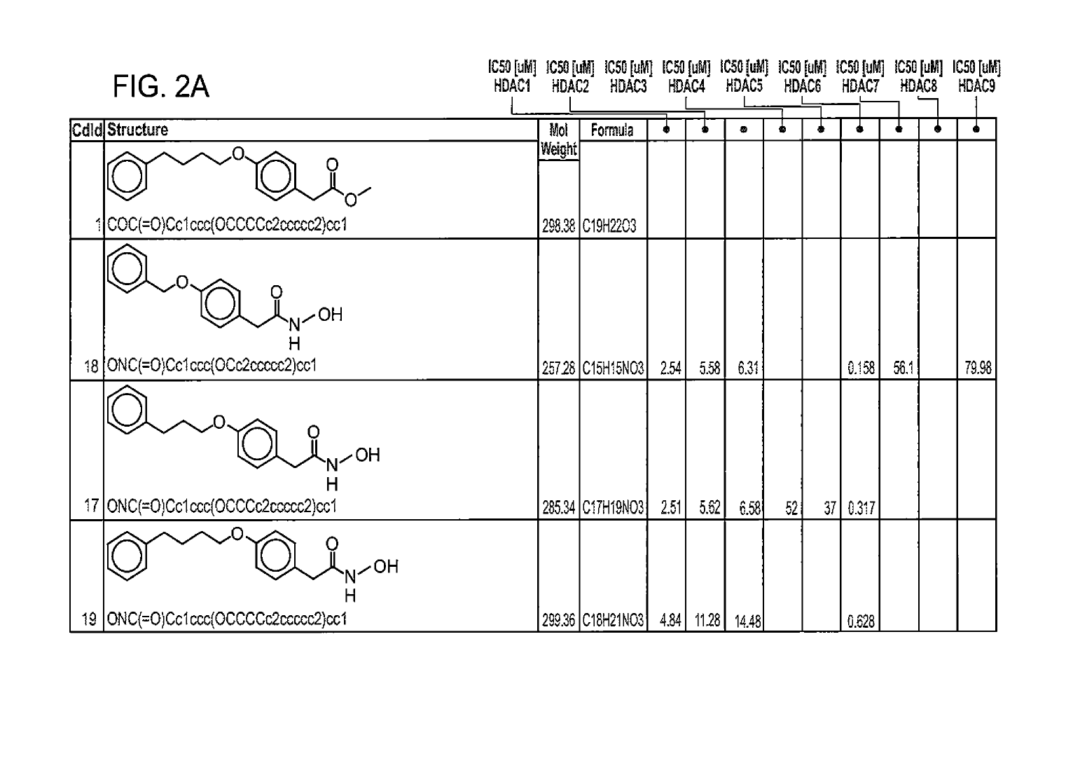

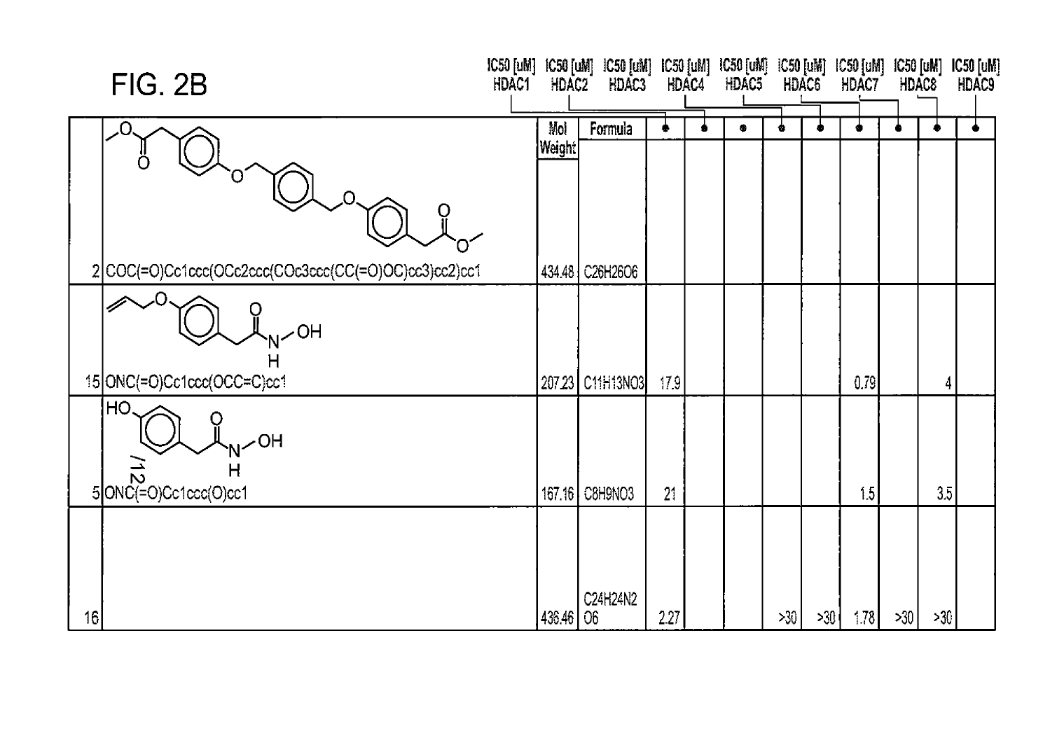

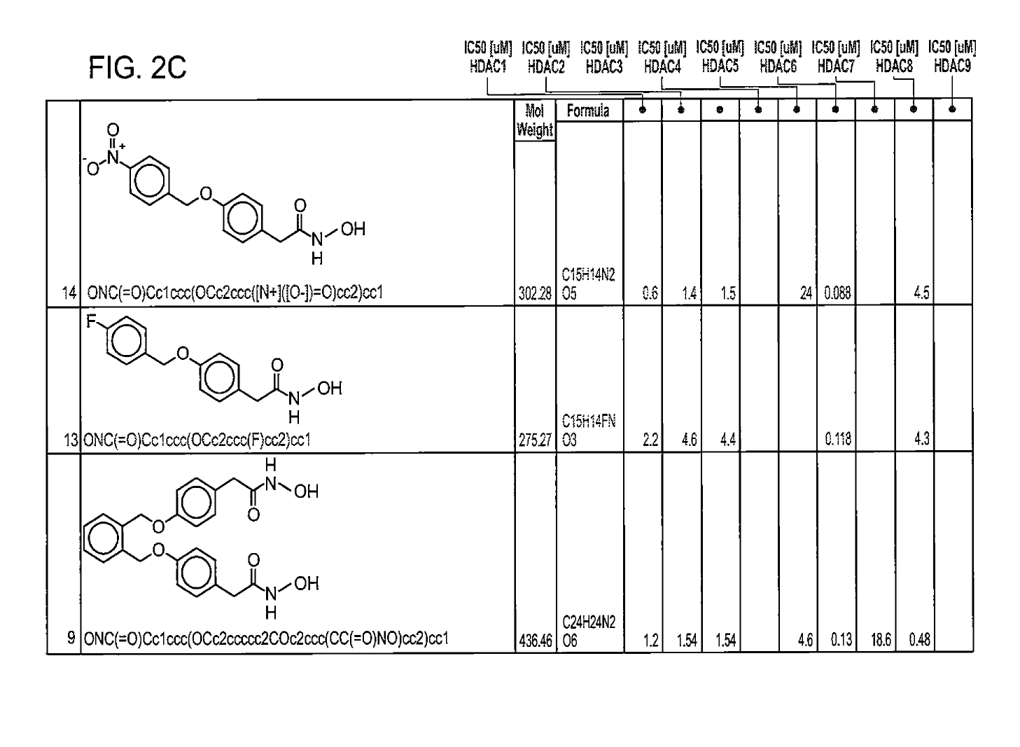

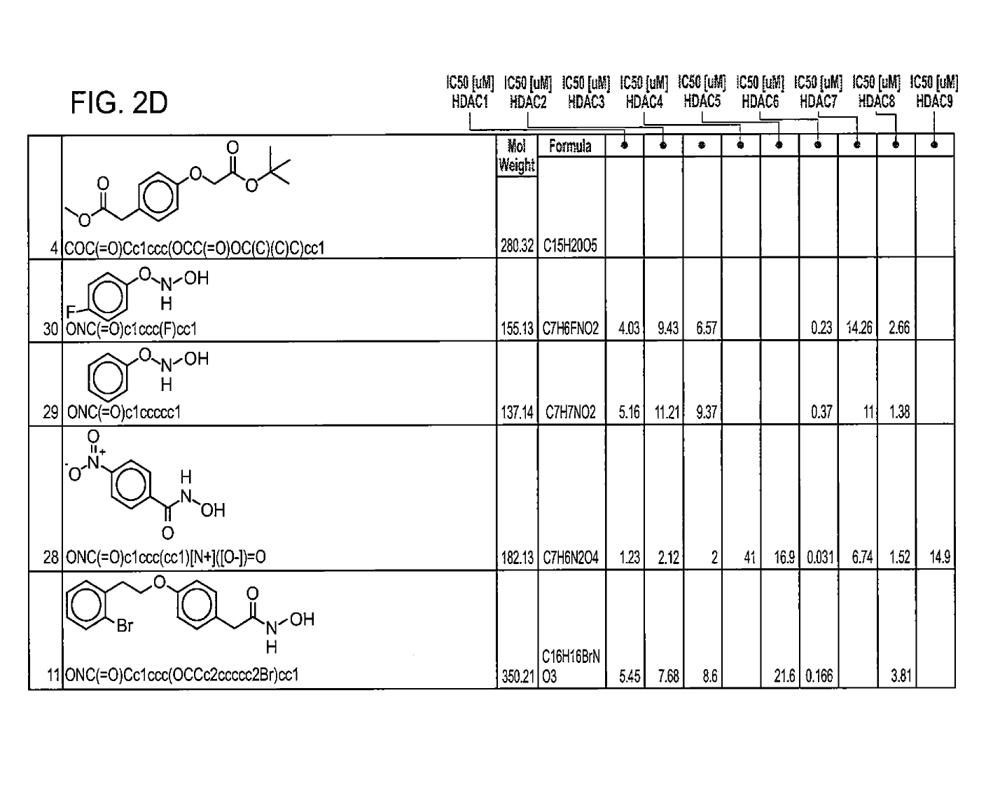

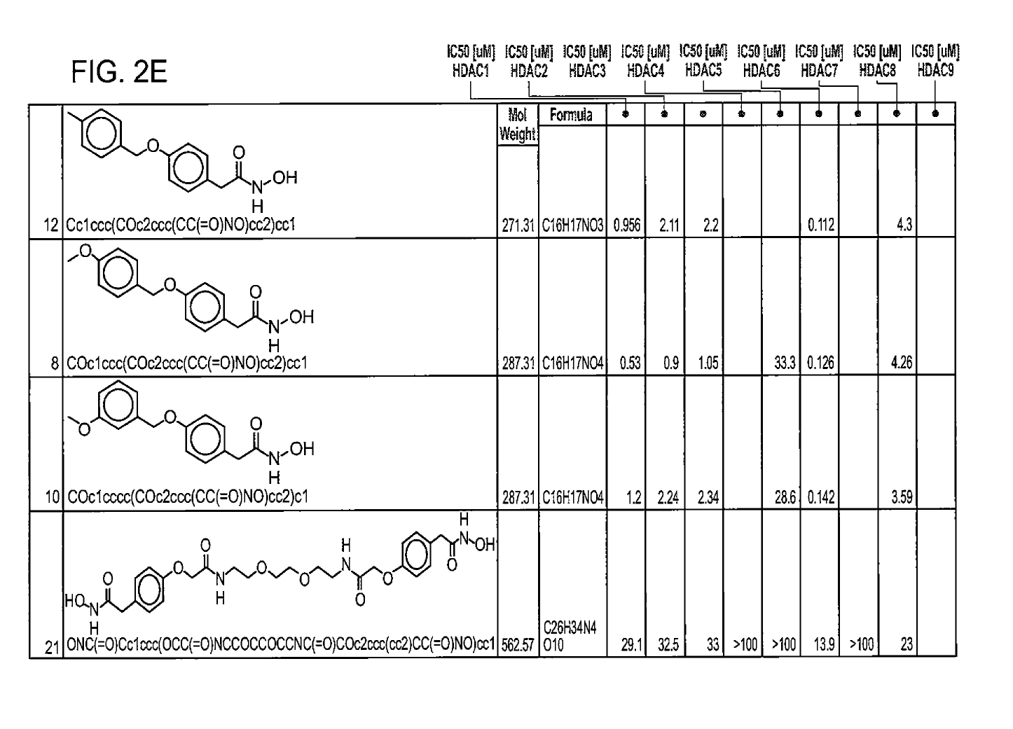

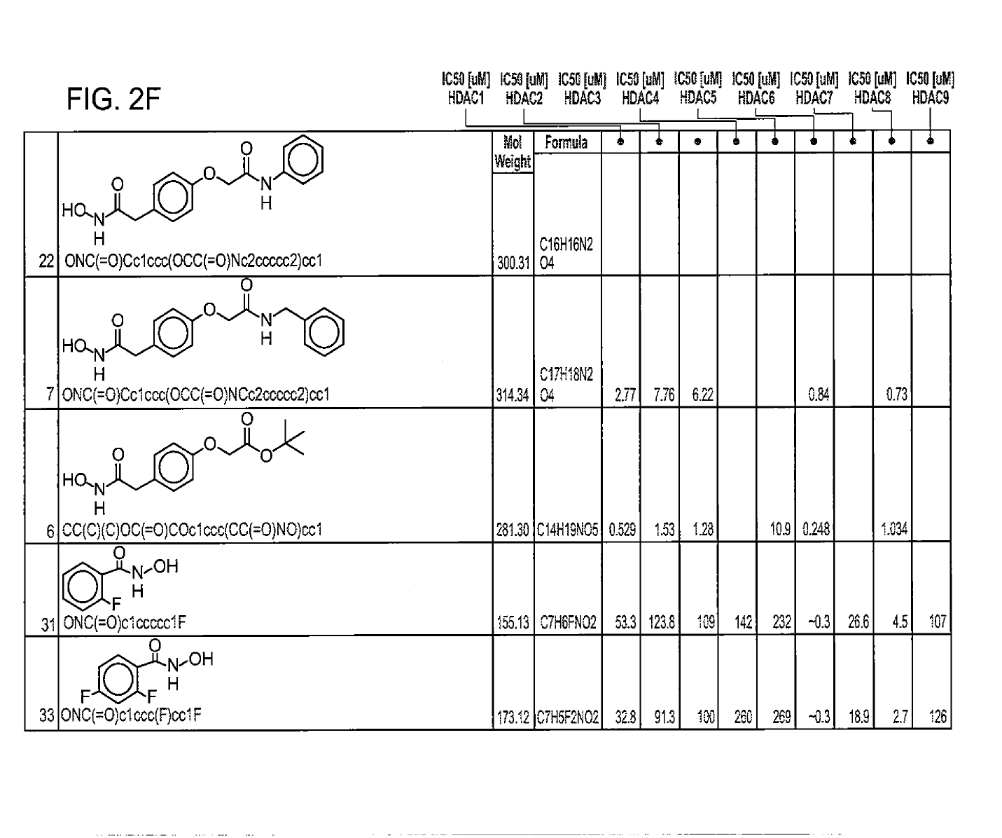

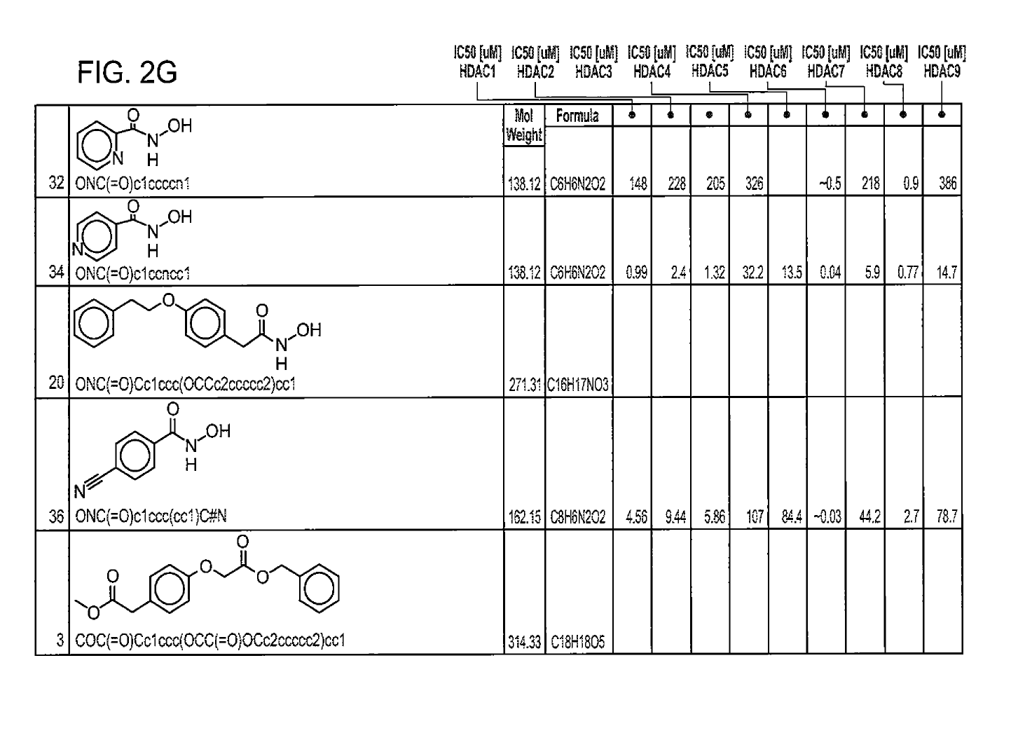

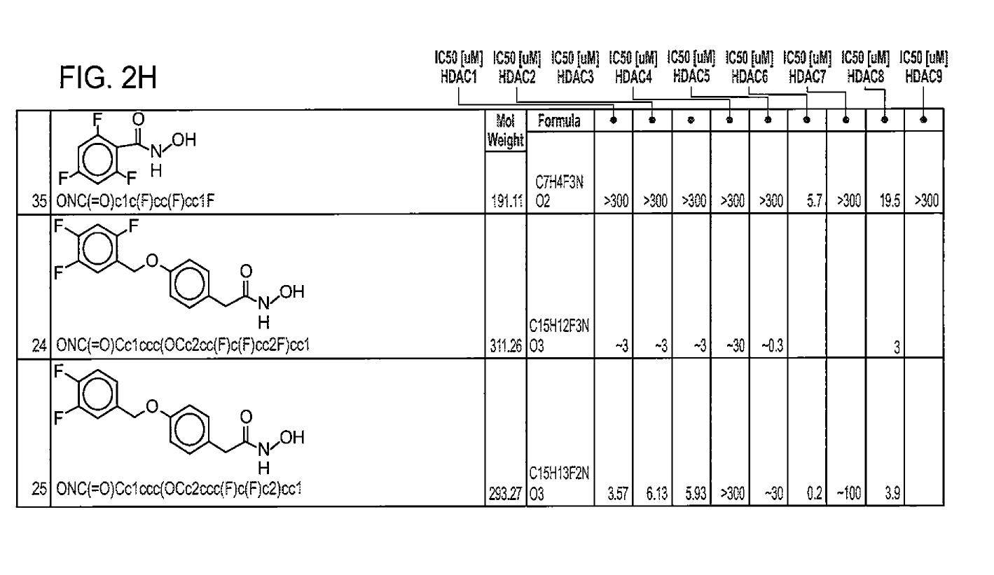

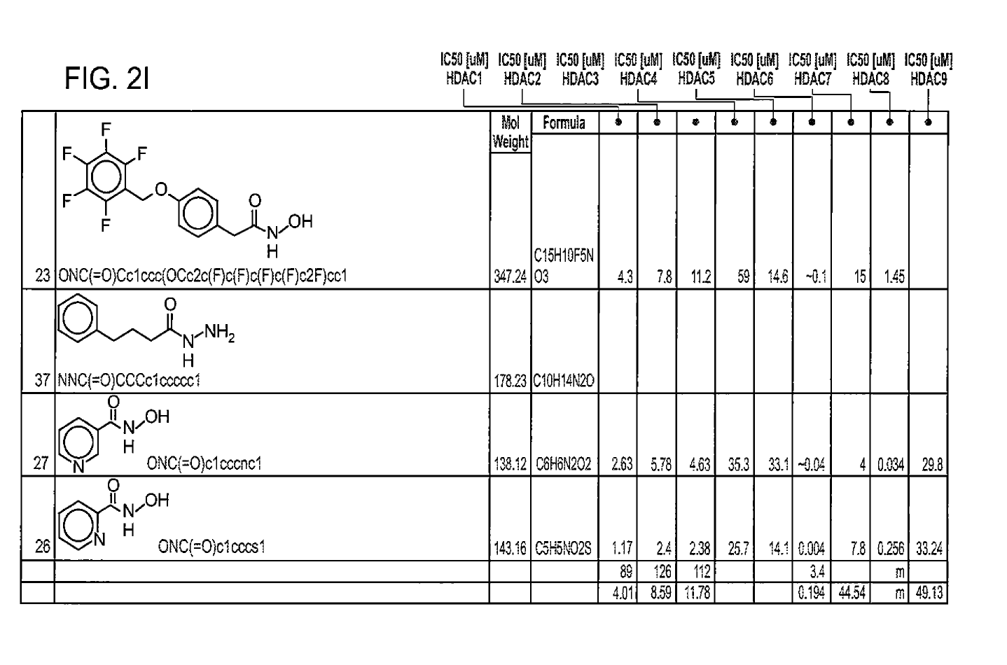

The molecular weights and other data associated with certain compounds results are shown in FIG. 2.

### Example 2

### Synthesis of Hydrazone-Linked Compounds General Description of Synthetic Methods

Certain compounds of the invention of Formula (I) or (III) containing a hydrazone ether linker between the cap (CAP) and the aryl group Ar can be prepared by reaction of a hydrazide with an aldehyde or ketone, e.g., as shown in Scheme 1, below:

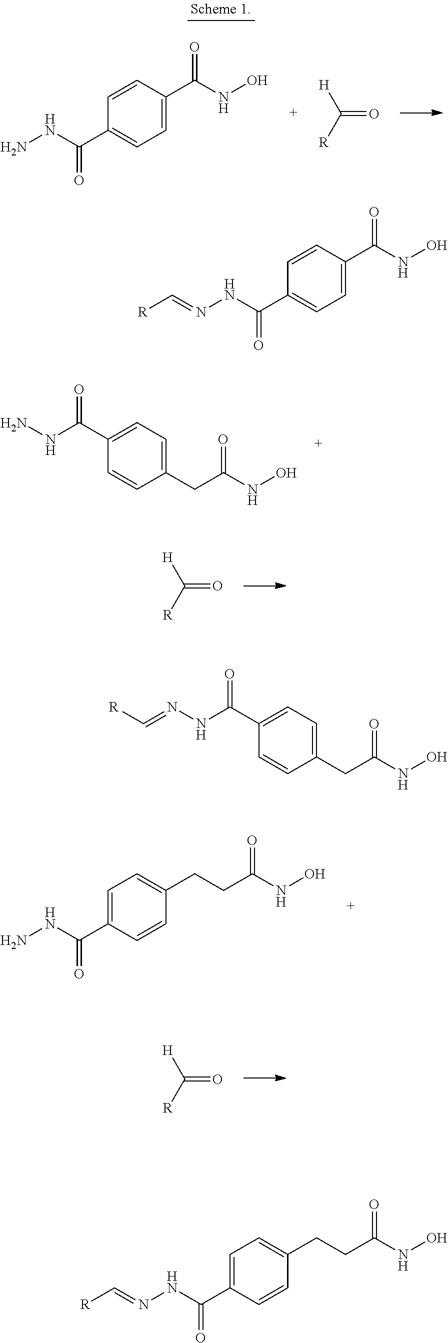

Exemplary aldehydes and ketones for reaction with hydrazides can be purchased commercially or can be prepared using a variety of methods, some of which are known in the art. The following aldehydes and ketones were used (i.e., “R” in each scheme of Scheme 1 can be, but is not limited to, the residue of any of the following aldehydes and ketones which were used for synthesis of hydrazone-containing compounds):

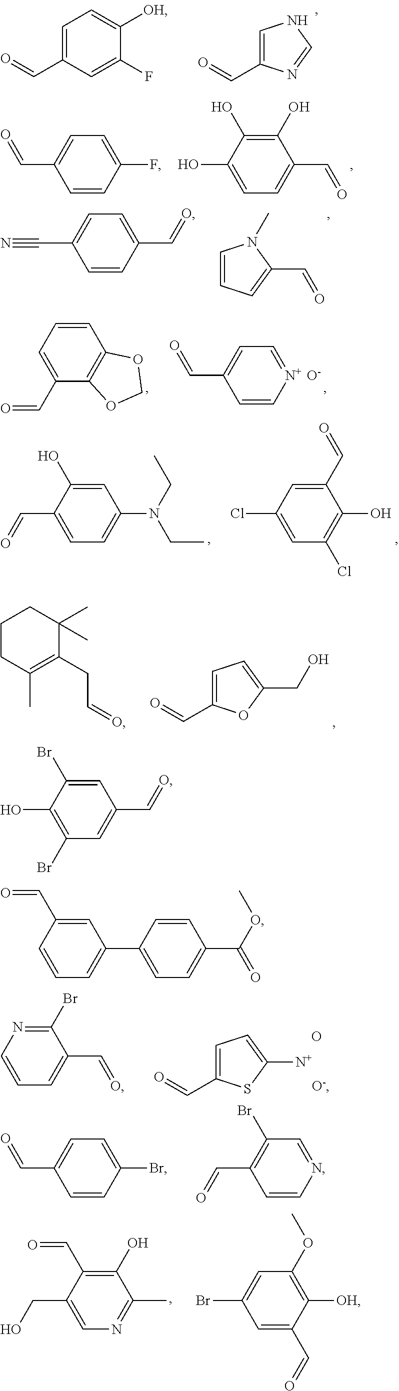

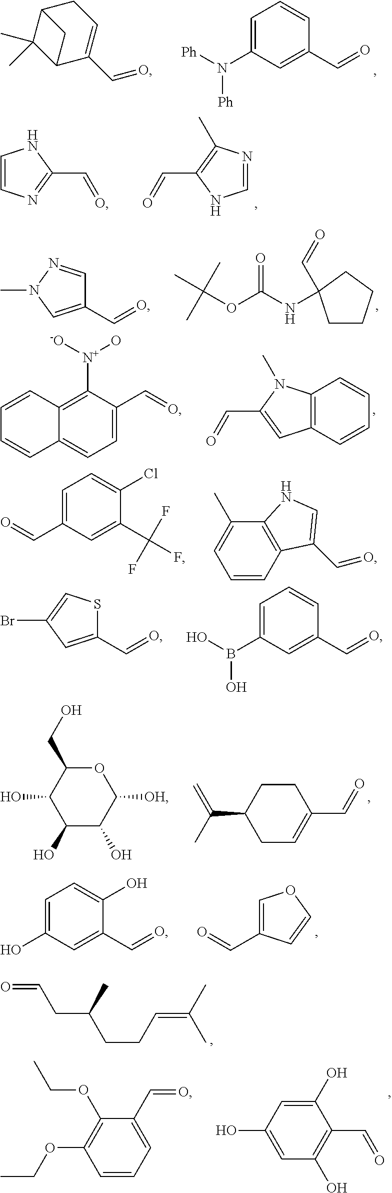

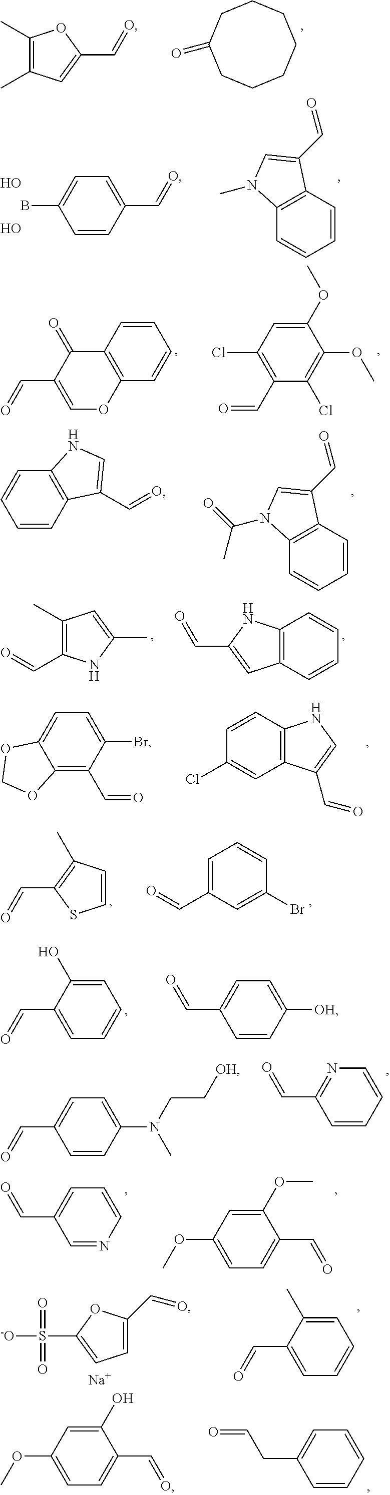

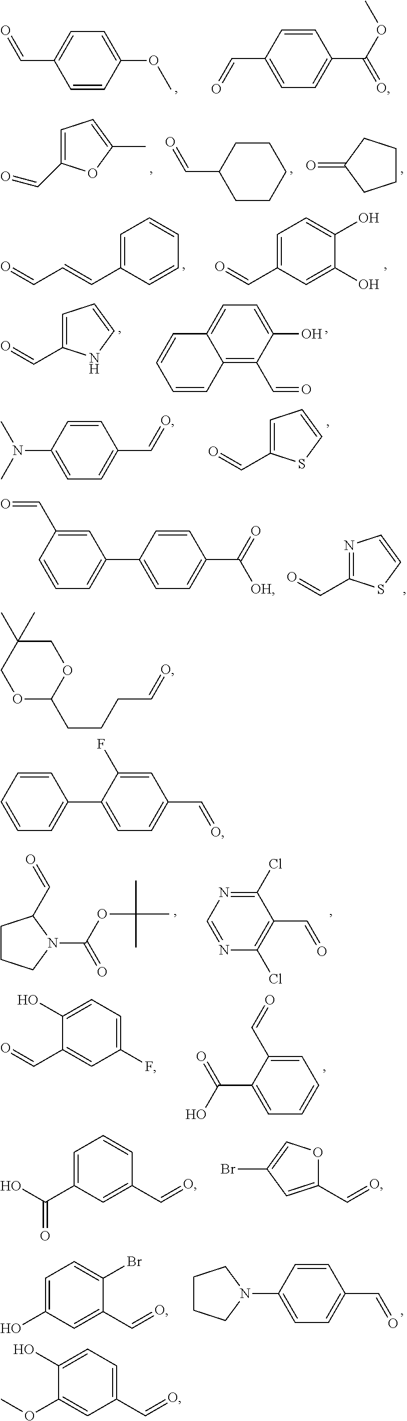

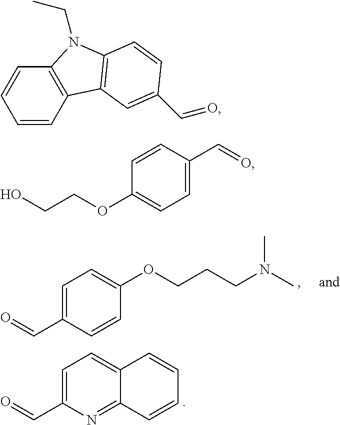

### Example 3

### Biological Activity of Exemplary Compounds

Selected compounds of the invention were tested for activity against a number of HDACs. The assays were performed substantially as described in PCT Patent Application WO/2008/091349. In brief, a target substrate is incubated with an HDAC or other protein with deacetylase activity in the presence of a test compound. In certain embodiments, the target substrate includes an acetylated lysine residue. Deacetylation of the substrate allows trypsin or another protease to cleave the substrate, thereby releasing a fluorescent probe. Fluorescence can be continuously monitored in order to determine the ability of the test compound to inhibit the deacetylation activity. The HDAC or other enzyme may be purified or used crude in the inventive assay. In certain embodiments, the assay is a cell-free assay.

In general, the compounds of the invention showed activity against one or more HDACs. Certain results are shown in FIG. 2. Certain preferred compounds had an IC₅₀ for HDAC6 of less than 1 μm, more preferably less than 100 nm, and even more preferably, less than 50 nm.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A compound represented by the formula:
CAP-L-Ar—(CR₁R₂)ₙ—Z (I)
wherein
R₁ and R₂ are each independently selected from the group consisting of H, halogen, C₁-C₄alkyl, or C₁-C₄haloalkyl;
Ar is a substituted or unsubstituted aryl group having from 5-14 atoms in the aryl ring(s);
L is a linker;
CAP is a cap;
Z is a chelator capable of binding Zn²⁺; and
n is 1, 2 or 3;
provided that if n is 1, CAP is not carbazolyl;
or a pharmaceutically acceptable salt thereof.

2. The compound of claim 1, wherein R₁ and R₂ are each H.

3. The compound of claim 1, wherein Ar is a substituted or unsubstituted carbocyclic aryl group having from 6 to 10 atoms in the rings system.

4. The compound of claim 1, wherein Ar is substituted or unsubstituted phenyl.

5. The compound of claim 1, wherein Ar is phenyl substituted L at the position on the phenyl ring para to the —(CR₁R₂)ₙ—Z moiety.

6. The compound of claim 1, wherein L is a linker having 1-6 atoms.

7. The compound of claim 6, wherein the linker comprises an alkylidene, an ether, a thioether, an amine, an amide, an ester, a carbonate, a carbamate, or a hydrazone.

8. The compound of claim 7, wherein the linker comprises —(CH₂)ₘ—O—, wherein m is an integer from 1 to 4.

9. The compound of claim 1, wherein CAP is a substituted or unsubstituted aryl group.

10. The compound of claim 9, wherein CAP is a substituted or unsubstituted phenyl group.

11. The compound of claim 10, wherein the phenyl group is substituted with 1-5 electron-withdrawing substituents.

12. The compound of claim 11, wherein the 1-5 electron-withdrawing substituents are 1-5 fluorine atoms.

13. The compound of claim 1, wherein Z is —C(O)NHOH.

14. The compound of claim 1, where in n is 1.

15. The compound of claim 1, wherein n is 2 or 3.

16. The compound of claim 1, wherein the compound is selected from the group consisting of:

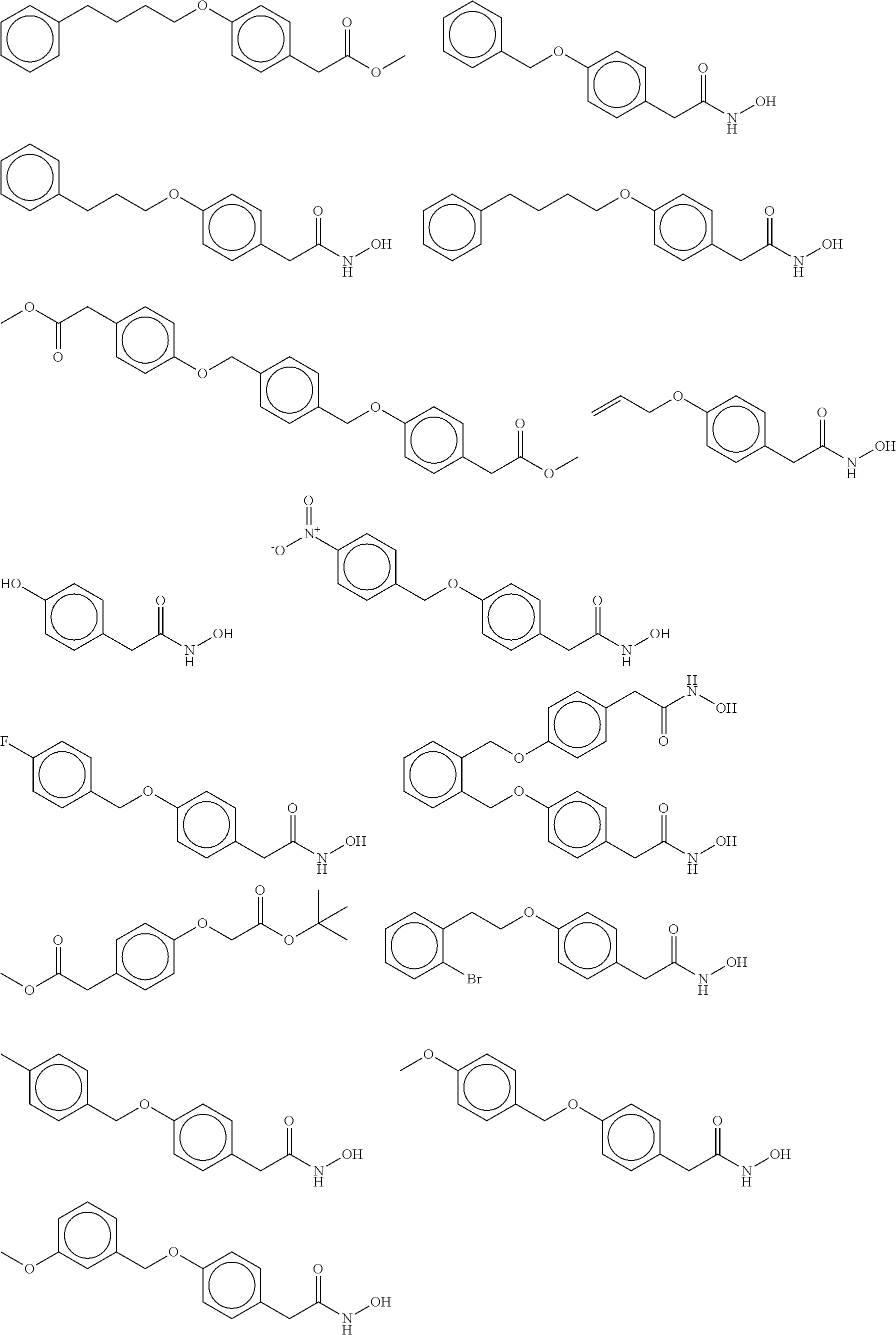

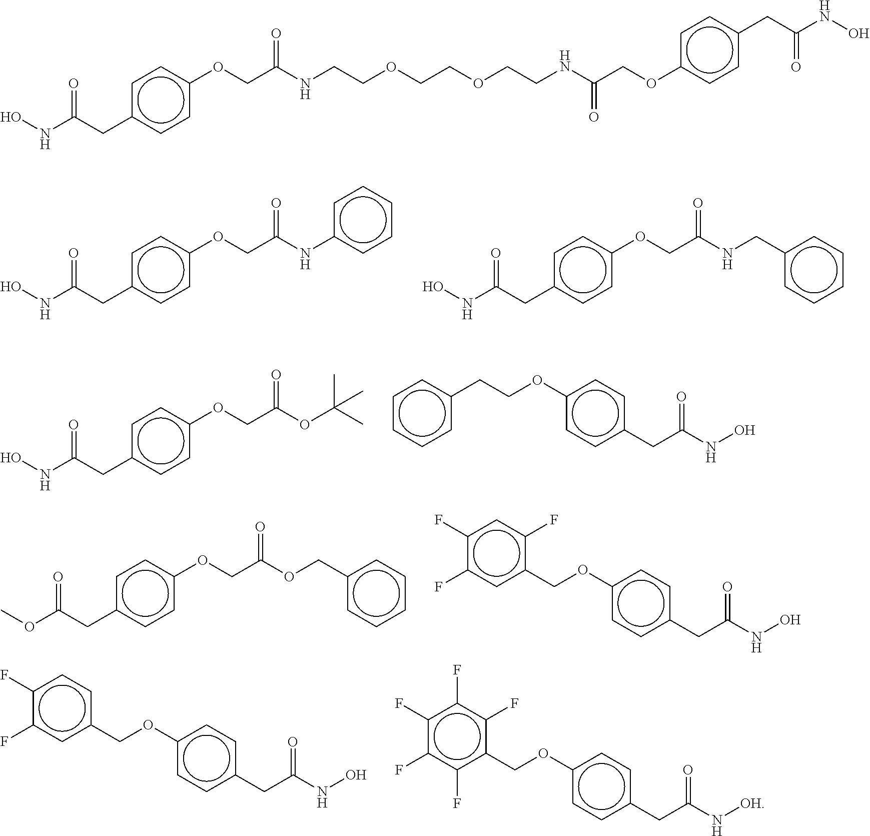

17. A compound represented by the formula:
Ar—C(O)NHOH (II)
wherein
Ar is a substituted or unsubstituted aryl or heteroaryl group having from 5-14 atoms in the aryl ring(s);
provided that if Ar is a phenyl group, the phenyl group is substituted;
or a pharmaceutically acceptable salt thereof.

18. The compound of claim 17, wherein Ar is a substituted phenyl group.

19. The compound of claim 17, wherein Ar is a substituted or unsubstituted pyridyl group.

20. The compound of claim 17, wherein Ar is a substituted or unsubstituted thiophenyl group.

21. The compound of claim 17, wherein Ar is a phenyl, pyridyl, or thiophenyl group substituted with one to three substituents selected from the group consisting of C₁-C₆alkyl, C₂-C₆alkenyl, C₂-C₆alkynyl, C₁-C₆haloalkyl, C₁-C₆hydroxyalkyl, C₁-C₆alkoxy, C₁-C₆alkylthio, F, Cl, Br, I, —OH, —NO₂, —CN, C₁-C₆alkylsulfonyl, —C(O)C₁-C₆alkyl, —CO₂(C₁-C₆alkyl), —CON(Rₓ)₂; —OC(O)C₁-C₆alkyl; —OCO₂ (C₁-C₆alkyl), —OCON(C₁-C₆alkyl)₂; —N(C₁-C₆alkyl)₂, and —NC₁-C₆alkyl(CO)C₁-C₆alkyl.

22. The compound of claim 21, wherein Ar is substituted with one substituent.

23. The compound of claim 22, wherein Ar is substituted with one substituent at a position ortho or para to the hydroxamate group.

24. The compound of claim 21, wherein at least one substituent is an electron-withdrawing group.

25. The compound of claim 24, wherein at least one substituent is selected from the group consisting of —NO₂, —CN, or F.

26. The compound of claim 17, wherein the compound is selected from the group consisting of:

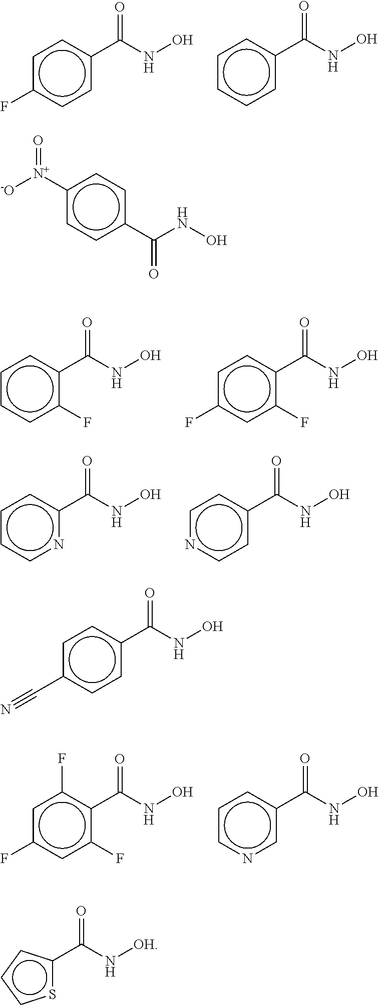

27. A compound represented by the formula:
CAP-L-Ar—C(O)NHOH (III)
wherein
Ar is an aryl or heteroaryl group having from 5-14 atoms in the aryl ring(s);
L is a linker;
CAP is a cap;
or a pharmaceutically acceptable salt thereof.

28. The compound of claim 27, wherein L is a linker having 1-6 atoms.

29. The compound of claim 28, wherein the linker comprises an alkylidene, an ether, a thioether, an amine, an amide, an ester, a carbonate, a carbamate, or a hydrazone.

30. The compound of claim 28, wherein the linker comprises —(CH₂)ₘ—O—, wherein m is an integer from 1 to 4.

31. The compound of claim 27, wherein CAP is a substituted or unsubstituted aryl group.

32. The compound of claim 31, wherein CAP is a substituted or unsubstituted phenyl group.

33. The compound of claim 32, wherein the phenyl group is substituted with 1-5 electron-withdrawing substituents.

34. The compound of claim 33, wherein the 1-5 electron-withdrawing substituents are 1-5 fluorine atoms.

35. A pharmaceutical composition comprising a therapeutically effective amount of a compound of claim 1, and a pharmaceutically acceptable excipient.

36. A method of inhibiting a deacetylase activity, the method comprising steps of contacting a deacetylase with a compound of claim 1.

37. The method according to claim 36, wherein the deacetylase is purified.

38. The method according to claim 37, wherein the deacetylase is in a cell.

39. The method according to claim 36, wherein the deacetylase is HDAC6 or HDAC8.

40. A method of treating a subject suffering from or susceptible to a proliferative disorder or a parasitic disease, the method comprising steps of administering a therapeutically effective amount of a compound of claim 1 to the subject such that the proliferative disorder or parasitic disease is treated or prevented.

41. The method according to claim 40, wherein the subject is a mammal.

42. The method according to claim 41, wherein the subject is human.

43. The method according to claim 40, wherein the proliferative disorder is cancer.

44. The method according to claim 43, wherein the proliferative disorder is an inflammatory disease.

45. The method according to claim 40 wherein the proliferative disorder is a proliferative disorder associated with the skin.

46. The method according to claim 40, wherein the proliferative disorder is cutaneous T-cell lymphoma.

47. The method according to claim 40, wherein the step of administering comprises administering the compound orally or intravenously.

48. A method of treating a subject suffering from or susceptible to a proliferative disorder or a parasitic disease, the method comprising steps of administering a therapeutically effective amount of bufexamac to the subject such that the proliferative disorder or parasitic disease is treated or prevented.

49. A packaged pharmaceutical comprising a therapeutically effective amount of a compound of claim 1, and written instructions for administration of the compound to a subject in need thereof.
